(19) United States
(12) Patent Application Publication
(10) Pub. No.: US 2022/0010274 A1
(43) Pub. Date: Jan. 13, 2022

(54) PERSONALIZED NEOANTIGEN-SPECIFIC ADOPTIVE CELL THERAPIES

(71) Applicant: PACT PHARMA, INC., South San Francisco, CA (US)

(72) Inventors: Barbara Sennino, San Francisco, CA (US); Kyle Jacoby, Emeryville, CA (US); Susan Foy, South San Francisco, CA (US); Ines Mende, South San Francisco, CA (US); Stefanie Mandl-Cashman, San Francisco, CA (US)

(73) Assignee: PACT PHARMA, INC., South San Francisco, CA (US)

(21) Appl. No.: 17/487,411

(22) Filed: Sep. 28, 2021

Related U.S. Application Data
(63) Continuation of application No. PCT/US2020/025758, filed on Mar. 30, 2020.
(60) Provisional application No. 62/826,824, filed on Mar. 29, 2019.

(51) Int. Cl. C12N 5/0783; C07K 14/725; A61K 35/17

(52) U.S. Cl. CPC C12N 5/0636; C07K 14/7051; C12N 2501/2307; C12N 2501/2302; A61K 35/17

(57) ABSTRACT

Methods of genetically engineering NeoTCR Products comprising young T cells and methods of manufacturing such cell products.

### CROSS-REFERENCE TO RELATED APPLICATIONS

The present application is a Continuation of International Patent Application No. PCT/US2020/025758, filed Mar. 30, 2020, which claims the benefit of priority of U.S. Provisional Application No. 62/826,824, filed Mar. 29, 2019, the contents of each of which are incorporated by reference herein in their entireties, and to each of which priority is claimed.

### BACKGROUND OF THE INVENTION

Clinical benefit observed with immuno-oncology trials often depends on the unleashing of a pre-existing intrinsic T cell immune response in each cancer patient. The targets of these intrinsic T cells are commonly ascribed to recognition of patient-specific neoantigens that arose from cancer mutations.

Methods used to engineer cells for adoptive cell therapies (ACT) utilizing receptors that are constant across many patients (CAR or shared Ag TCRs) typically rely on Lenti-, retro-, or adeno-associated virus to deliver specificity-altering sequences to T cells. However, for personalized therapies such as the generation of neoepitope-specific TCR T cell therapies, use of viral vectors is not feasible due to long manufacturing timelines and prohibitive per-patient costs.

Furthermore, another limitation of ACT is persistence of the engineered cells in a patient following infusion. Current ACTs have a limited persistence following infusion. This results in multiple and consecutive infusions of the ACT and the inability of the ACT to implant into the patient to become a memory T cell.

What is needed, therefore, are improved methods and compositions for manufacturing of engineered cells for improved targeted personalized therapies.

### SUMMARY OF THE INVENTION

The methods and compositions described herein enable personalized, autologous neo-epitope specific TCR-engineered T cell therapies for the eradication of solid and liquid tumors. In some embodiments, these therapies are facilitated by selective capture neoantigen-specific CD8 T cells from peripheral blood of the patient.

In some embodiments, provided herein are highly efficient, DNA-mediated (non-viral) precision genome engineering methods to engineer neoepitope-specific primary human T cells. These methods can be widely utilized to generate T cells at research scale, as well as for ex vivo manufacturing.

In some embodiments, genomes of individual primary human CD8 and CD4 T cells are engineered with site-specific nucleases in a single-step transfection process to yield efficient, targeted replacement of the endogenous TCR with the therapeutic neoTCR sequences. In this way, the expression of the endogenous TCR is abolished ensuring natural expression and regulation of the inserted neoTCR.

In some embodiments, using the imPACT Isolation Technology described in PCT/US2020/17887 (which is herein incorporated by reference in its entirety), neoepitope-specific TCRs were cloned and autologous CD8+ and CD4+ T cells from the same patient with cancer are precision genome engineered (using a DNA-mediated (non-viral) method) to express the neoTCR. NeoTCR expressing T cells are then expanded in a manner that preserves a “younger” T cell phenotypes, resulting in a NeoTCR Product in which the majority of the T cells exhibit T memory stem cell and T central memory phenotypes.

The genome engineering approach described herein enables highly efficient generation of bespoke NeoTCR T cells for personalized adoptive cell therapy for patients with solid and liquid tumors. Furthermore, the engineering method is not restricted to the use in T cells and has also been applied successfully to other primary cell types, including natural killer and hematopoietic stem cells.

In certain embodiments, the presently disclosed subject matter provides a method of producing a population of modified young T cells, comprising a) introducing into a T cell a homologous recombination (HR) template nucleic acid sequence comprising i) first and second homology arms homologous to first and second target nucleic acid sequences; ii) a TCR gene sequence positioned between the first and second homology arms; b) recombining the HR template nucleic acid into an endogenous locus of the cell comprising the first and second endogenous sequences homologous to the first and second homology arms of the HR template nucleic acid; and c) culturing the T cell to produce a population of young T cells.

In certain embodiments, the HR template comprises a first 2A-coding sequence positioned upstream of the TCR gene sequence and a second 2A-coding sequence positioned downstream of the TCR gene sequence, wherein the first and second 2A-coding sequences code for the same amino acid sequence that are codon-diverged relative to each other. In certain embodiments, the 2A-coding sequence is a P2A-coding sequence.

In certain embodiments, a sequence coding for the amino acid sequence Gly Ser Gly is positioned immediately upstream of the 2A-coding sequences. In certain embodiments, the HR template comprises a sequence coding for a Furin cleavage site positioned upstream of the second 2A-coding sequence.

In certain embodiments, the first and second homology arms are each from about 300 bases to about 2,000 bases in length. In certain embodiments, the first and second homology arms are each from about 600 bases to about 2,000 bases in length.

In certain embodiments, the HR template further comprises a signal sequence positioned between the first 2A-coding sequence and the TCR gene sequence.

In certain embodiments, the HR template comprises a second TCR sequence positioned between the second 2A-coding sequence and the second homology arm.

In certain embodiments, the HR template comprises a) a first signal sequence positioned between the first 2A-coding sequence and the first TCR gene sequence; and b) a second signal sequence positioned between the second 2A-coding sequence and the second TCR gene sequence; wherein the first and the second signal sequences encode for the same amino acid sequence and are codon diverged relative to each other.

In certain embodiments, the signal sequence is a human growth hormone signal sequence.

In certain embodiments, the HR template is non-viral. In certain embodiments, the TCR template is a circular DNA. In certain embodiments, the TCR template is a linear DNA.

In certain embodiments, the T cell is a patient-derived cell.

In certain embodiments, the endogenous locus is within an endogenous TCR gene.

In certain embodiments, the TCR gene sequence encodes for a TCR that recognizes a tumor antigen.

In certain embodiments, the tumor antigen is a neoantigen. In certain embodiments, the tumor antigen is a patient specific neoantigen.

In certain embodiments, the TCR gene sequence is a patient specific TCR gene sequence.

In certain embodiments, said recombining comprises a) cleavage of the endogenous locus by a nuclease; and b) recombination of the HR template nucleic acid sequence into the endogenous locus by homology directed repair.

In certain embodiments, the nuclease is a Clustered Regularly Interspaced Short Palindromic Repeats (CRISPR) family nuclease or derivative thereof. In certain embodiments, nuclease further comprises an sgRNA.

In certain embodiments, the introducing occurs via electroporation.

In certain embodiments, the culturing is conducted in the presence of at least one cytokine. In certain embodiments, the culturing is conducted in the presence of IL2, IL7, IL15, or any combination thereof. In certain embodiments, the culturing is conducted in the presence of IL7 and IL15.

In certain embodiments, the population of young T cells comprises cells that are CD45RA+, CD62L+, CD28+, CD95−, CCR7+, and CD27+. In certain embodiments, the population of young T cells comprises cells that are CD45RA+, CD62L+, CD28+, CD95+, CD27+, CCR7+. In certain embodiments, the population of young T cells comprises cells that are CD45RO+, CD62L+, CD28+, CD95+, CCR7+, CD27+, CD127+.

In certain embodiments, the population of young T cells maintains its killing activity for at least about 14 days-60 days. In certain embodiments, the population of young T cells maintains its killing activity for at least about 14 days, at least about 21 days, at least about 28 days, at least about 35 days, at least about 42 days, at least about 49 days, at least about 56 days, at least about 63 days, at least about 70 days, at least about 77 days, at least about 84 days, at least about 91 days, at least about 98 days, at least about 105 day, or at least about 112 days. In certain embodiments, the population of young T cells maintains its killing activity for at least about 61 days-120 days. In certain embodiments, the population of young T cells maintains its killing activity for more than 120 days.

In certain embodiments, the presently disclosed subject matter provides a population of young T cells obtained by the any of the methods disclosed herein.

In certain embodiments, the presently disclosed subject matter provides a pharmaceutical composition comprising the population of young T cells obtained by any of the methods disclosed herein. In certain embodiments, the pharmaceutical composition is administered to a patient in need thereof for the treatment of cancer, and wherein cells of the composition engraft in the patient as Tmsc or Tcm cells.

In certain embodiments, the presently disclosed subject matter provides a method of treating cancer in a subject in need thereof, the method comprising a) modifying patient-derived T cells by introducing a homologous recombination (HR) template into the T cell, wherein the HR template comprises i) first and second homology arms homologous to first and second target nucleic acid sequences; ii) a TCR gene sequence positioned between the first and second homology arms; b) recombining the polynucleotide into an endogenous locus of the T cell; c) culturing the modified T cell to produce a population of young T cells; and d) administering a therapeutically effective amount of the population of modified young T cells to the human patient to thereby treat the cancer.

In certain embodiments, a non-myeloablative lymphodepletion regimen is administered to the subject prior to administering a therapeutically effective amount of modified young T cells.

In certain embodiments, the cancer is a solid tumor. In certain embodiments, the cancer is a liquid tumor. In certain embodiments, the solid tumor selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, and colorectal cancer. In certain embodiments, the liquid tumor is selected from the group consisting of follicular lymphoma, leukemia, and multiple myeloma.

In certain embodiments, the HR template comprises a first 2A-coding sequence positioned upstream of the TCR gene sequence and a second 2A-coding sequence positioned downstream of the TCR gene sequence, wherein the first and second 2A-coding sequences code for the same amino acid sequence that are codon-diverged relative to each other.

In certain embodiments, the 2A-coding sequence is a P2A-coding sequence.

In certain embodiments, a sequence coding for the amino acid sequence Gly Ser Gly is positioned immediately upstream of the 2A-coding sequences.

In certain embodiments, the HR template comprises a sequence coding for a Furin cleavage site positioned upstream of the second 2A-coding sequence.

In certain embodiments, the first and second homology arms are each from about 300 bases to about 2,000 bases in length. In certain embodiments, the first and second homology arms are each from about 600 bases to about 2,000 bases in length.

In certain embodiments, the HR template further comprises a signal sequence positioned between the first 2A-coding sequence and the TCR gene sequence. In certain embodiments, the HR template comprises a second TCR sequence positioned between the second 2A-coding sequence and the second homology arm.

In certain embodiments, the HR template comprises: a first signal sequence positioned between the first 2A-coding sequence and the first TCR gene sequence; and a second signal sequence positioned between the second 2A-coding sequence and the second TCR gene sequence; wherein the first and the second signal sequences encode for the same amino acid sequence and are codon diverged relative to each other.

In certain embodiments, the signal sequence is a human growth hormone signal sequence.

In certain embodiments, the HR template is non-viral.

In certain embodiments, the HR template is a circular DNA. In certain embodiments, the HR

template is a linear DNA.

In certain embodiments, the endogenous locus is within an endogenous TCR gene.

In certain embodiments, the TCR gene sequence encodes for a TCR that recognizes a tumor antigen.

In certain embodiments, the tumor antigen is a neoantigen. In certain embodiments, the tumor antigen is a patient specific neoantigen.

In certain embodiments, the TCR gene sequence is a patient specific TCR gene sequence.

In certain embodiments, said recombining comprises cleavage of the endogenous locus by a nuclease; and recombination of the HR template nucleic acid sequence into the endogenous locus by homology directed repair.

In certain embodiments, the nuclease is a Clustered Regularly Interspaced Short Palindromic Repeats (CRISPR) family nuclease or derivative thereof. In certain embodiments, the nuclease further comprises an sgRNA.

In certain embodiments, the introducing occurs via electroporation.

In certain embodiments, the culturing is conducted in the presence of at least one cytokine. In certain embodiments, the culturing is conducted in the presence of IL2, IL7, IL15, or any combination thereof. In certain embodiments, the culturing is conducted in the presence of IL7 and IL15.

In certain embodiments, the population of young T cells comprises cells that are CD45RA+, CD62L+, CD28+, CD95−, CCR7+, and CD27+. In certain embodiments, the population of young T cells comprises cells that are CD45RA+, CD62L+, CD28+, CD95+, CD27+, CCR7+. In certain embodiments, the population of young T cells comprises cells that are CD45RO+, CD62L+, CD28+, CD95+, CCR7+, CD27+, CD127+.

In certain embodiments, the population of young T cell maintains its killing activity for at least about 14 days. In certain embodiments, the population of young T cells maintains its killing activity for at least about 60 days, or between 60 days and 120 days, or 121 days and 180 days, or 181 days and 250 days, or 251 days and 365 days, or greater than one year.

In certain embodiments, the population of young T cells engrafts into the patient following the administration of a therapeutically effective amount of the population of modified young T cells.

In certain embodiments, the engrafted cells activate upon neoantigen presentation on a tumor cell. In certain embodiments, the engrafted cells kill the tumor cell.

In certain embodiments, the engrafted cells can activate and kill a tumor cell for up to 30 days, for 31-60 days, for 61-90 days, for 91-180 days, for 181-250 days, for 251-265 days, or for over 1 year following administration to the patient.

In certain embodiments, the presently disclosed subject matter provides a method of modifying a cell, wherein the cell is a natural killer cell or hematopoietic stem cell, the method comprising a) introducing into the cell a homologous recombination (HR) template nucleic acid sequence comprising i) first and second homology arms homologous to first and second target nucleic acid sequences; ii) a TCR gene sequence positioned between the first and second homology arms; b) recombining the HR template nucleic acid into an endogenous locus of the cell comprising the first and second endogenous sequences homologous to the first and second homology arms of the HR template nucleic acid.

In certain embodiments, the HR template comprises a first 2A-coding sequence positioned upstream of the TCR gene sequence and a second 2A-coding sequence positioned downstream of the TCR gene sequence, wherein the first and second 2A-coding sequences code for the same amino acid sequence that are codon-diverged relative to each other.

In certain embodiments, the 2A-coding sequence is a P2A-coding sequence. In certain embodiments, a sequence coding for the amino acid sequence Gly Ser Gly is positioned immediately upstream of the 2A-coding sequences.

In certain embodiments, the HR template comprises a sequence coding for a Furin cleavage site positioned upstream of the second 2A-coding sequence.

In certain embodiments, the first and second homology arms are each from about 300 bases to about 2,000 bases in length.

In certain embodiments, the HR template further comprises a signal sequence positioned between the first 2A-coding sequence and the TCR gene sequence.

In certain embodiments, the HR template comprises a second TCR sequence positioned between the second 2A-coding sequence and the second homology arm.

In certain embodiments, the HR template comprises a first signal sequence positioned between the first 2A-coding sequence and the first TCR gene sequence; and a second signal sequence positioned between the second 2A-coding sequence and the second TCR gene sequence; wherein the first and the second signal sequences encode for the same amino acid sequence and are codon diverged relative to each other.

In certain embodiments, the signal sequence is a human growth hormone signal sequence.

In certain embodiments, the HR template is non-viral. In certain embodiments, the HR template is a circular DNA. In certain embodiments, the HR template is a linear DNA.

In certain embodiments, the cell is a patient-derived cell.

In certain embodiments, the endogenous locus is within an endogenous TCR gene.

In certain embodiments, the TCR gene sequence encodes for a TCR that recognizes a tumor antigen. In certain embodiments, the tumor antigen is a neoantigen. In certain embodiments, the tumor antigen is a patient specific neoantigen.

In certain embodiments, the TCR gene sequence is a patient specific TCR gene sequence.

In certain embodiments, said recombining comprises cleavage of the endogenous locus by a nuclease; and recombination of the HR template nucleic acid sequence into the endogenous locus by homology directed repair.

In certain embodiments, the nuclease is a Clustered Regularly Interspaced Short Palindromic Repeats (CRISPR) family nuclease or derivative thereof. In certain embodiments, the nuclease further comprises an sgRNA.

In certain embodiments, the presently disclosed subject matter provides a method wherein IL2 is not used in the culturing.

In certain embodiments, the presently disclosed subject matter provides a pharmaceutical formulation comprising young T cells made using any of the methods described herein, wherein the pharmaceutical formation comprises at least 20% Tmsc and Tcm collectively, at least 25% Tmsc and Tcm collectively, at least 30% Tmsc and Tcm collectively, at least 35% Tmsc and Tcm collectively, at least 40% Tmsc and Tcm collectively, at least 45% Tmsc and Tcm collectively, at least 50% Tmsc and Tcm collectively, at least 55% Tmsc and Tcm collectively, at least 60% Tmsc and Tcm collectively or more than 61% Tmsc and Tcm collectively.

In certain embodiments, the final formulation is cryopreserved in 46% Plasma-Lyte A, 1% HSA (w/v), and 50% CryoStor CS10.

### BRIEF DESCRIPTION OF THE DRAWINGS

This application contains at least one drawing executed in color. Copies of this patent or patent application publication with color drawing(s) will be provided by the Office upon request and payment of the necessary fee.

FIG. 1. FIG. 1 provides a high-level diagram of the knock-out and knock-in at the endogenous TCR locus accomplished by the gene editing technology described in Example 1.

FIGS. 2A-2C. FIGS. 2A-2C show an example of a NeoE TCR cassette and gene editing methods that can be used to make NeoTCR Products. FIG. 2A shows a schematic representing the general targeting strategy used for integrating neoantigen-specific TCR constructs (neoTCRs) into the TCRα locus. FIGS. 2B and 2C show a neoantigen-specific TCR construct design used for integrating a NeoTCR into the TCRα locus wherein the cassette is shown with signal sequences (“SS”), protease cleavage sites (“P”), and 2A peptides (“2A”). FIG. 2B shows a target TCRα locus (endogenous TRAC, top panel) and its CRISPR Cas9 target site (horizontal stripes, cleavage site designated by the arrow), and the circular plasmid HR template (bottom panel) with the polynucleotide encoding the neoTCR, which is located between left and right homology arms (“LHA” and “RHA” respectively) prior to integration. FIG. 2C shows the integrated neoTCR in the the TCRα locus (top panel), the transcribed and spliced neoTCR mRNA (middle panel), and translation and processing of the expressed neoTCR (bottom panel).

FIG. 3. FIG. 3 shows the results of an In-Out PCR confirming precise target integration of the NeoE TCR cassette. Agarose gels show the results of a PCR using primers specific to the NeoE TCR cassette and relative site generate products of the expected size only for cells treated with both nuclease and DNA template (knock-out-knock-in (KOKI) and knock-out-knock-in-knock-out (KOKIKO)), demonstrating site-specific and precise integration.

FIG. 4. FIG. 4 shows the results from the Targeted Locus Amplification (TLA) analysis that was used to confirm the specificity of targeted integration.

FIGS. 5A and 5B. FIG. 5A shows results from a FACS experiment showing that the endogenous TCR has reduced signal and that there is a strong NeoE TCR signal in cells that were electroporated with the NeoE TCR cassette. FIG. 5B shows the results from a series of multiple transfection experiments with the NeoE TCR cassette showing a high degree of reproducibility between experiments.

FIGS. 6A-6C. FIG. 6A shows that the expression of the NeoE TCR in cells electroporated with the NeoE TCR cassette is substantially similar to the endogenous TCR expression in non-electroporated or mock-electroporated cells. FIG. 6B shows that the expression of the NeoE TCR is independent of the NeoTCR selected for expression. Each of the NeoE TCRs (squares) specific for Neo12, MART1 (i.e. F5), or NY-ESO (i.e., 1G4) had similar expression rates to the endogenous TCR (circles). FIG. 6C shows the expression profile of NeoE TCR expressing cells on days 10 and 27 following transfection with a NeoE TCR cassette. The NeoE TCR expression shown in FIG. 6C was detected with dextramer staining and shows that the NeoE TCR expression persists in extended cell culture periods of time.

FIG. 7. FIG. 7 shows micrographs of cells in culture up to three days after transfection with a cassette comprising a NeoE TCR in tandem with mCherry protein. Time-lapse photography shows a high level of mCherry expression 2-3 days post-transfection.

FIGS. 8A and 8B. FIGS. 8A and 8B illustrate the characterization of the T cells described in the present disclosure and present data showing that the engineered NeoE T cells (i.e., NeoTCR Products) are highly functional as demonstrated by antigen-specific proliferation, killing, and cytokine production. FIG. 8A shows the total CD4 and CD8 T cell subset distribution on day 13 following manufacturing of the NeoTCR Product in healthy patients (full bar) and in cancer patients (empty bar). FIG. 8B shows that CD4 T cells (left panel) and CD8 T cells (right panel) have a predominant phenotype of Tmsc and Tem following expansion. Tmsc: memory T stem cells; Tem: central memory T cells; TtmTem: transitional memory T cells; Teff: effector memory T cells.

FIG. 9. FIG. 9 shows data from T cells that were engineered to express the Neo12 NeoE TCR (Neo12 T Cells) and were co-cultured with tumor cells expressing the cognate Neo12 peptide (K562 HLA-A2 + neo12). Upon exposure to the cognate antigen-expressing tumor cells, the Neo12 T Cells rapidly differentiated into potent effector T cells. Also , no changes were observed when the Neo12 T Cells were co-cultured with tumor cells lacking the cognate antigen (HLA tumor cells—control).

FIGS. 10A-10C. FIGS. 10A-10C show data from NeoTCR T cells that were engineered to express either the neo12 TCR (Neo12 T cells) or the F5 (MART1) TCR (F5 T cells). FIG. 10A shows that the Neo12 T cells and the F5 T cells showed functional activity as measured by antigen-specific IFNγ cytokine secretion. FIG. 10B shows that the Neo12 T cells and the F5 T cells showed functional activity as measured by antigen-specific target cell killing. FIG. 10C shows that the Neo12 TCR T cells and the F5 T cells showed functional activity as measured by proliferation.

FIGS. 11A and 11B. FIGS. 11A and 11B show that there is comparable antigen specific activity of NeoTCR Products made with T cells derived from patients with cancer and patients without cancer. FIG. 11A shows the percent of T cells transfected with the neo12 TCR wherein the T cells are either from cancer patients (“Patient”) or non-cancer patients (“Healthy”). FIG. 11B shows the functional activity of T cells transfected with the neo12 TCR in cells that were acquired from cancer patients (“Patient”) and non-cancer patients (“Healthy”). The functional activity is shown through a killing assay, a proliferation assay, and a cytokine production (IFNγ, IL2, and TNFα) that was measured from supernatant using a cytokine bead assay.

FIGS. 12A and 12B. FIGS. 12A and 12B show specific killing of antigen-espressing surrogate tumor target cells and antigen-specific proliferation of NeoTCR Products. FIG. 12A shows NeoTCR Products that express the neo12 NeoTCR and mCherry that were co-cultured with tumor cells expressing ZsGreen and the specific Neo12 antigen (with HLA complex). After encountering antigen-expressing tumor cells, the NeoTCR Product cells became elongated, formed immunological synapses, and killed the target tumor cell. Unshown data showed that non-gene edited cells (T cells that did not express the neo12 TCR) had no cytotoxic activity. FIG. 12B shos timelapse microscopy of tumor cell death and T cell proliferation of the NeoTCR Product in response to antigen-specific tumor cell encounter.

FIG. 13. FIG. 13 shows that CD4 and CD8 NeoTCR Products are polyfunctional.

FIG. 14. FIG. 14 shows that NeoTCR Products exhibit polyfunctional responses that are strongly driven by proteins associated with effector function.

FIGS. 15A-15C. FIG. 15A shows that the precision engineering used to gene edit T cells to make the NeoTCR Products can be applied to hematopoietic stem cells (HSCs). Specifically, HSCs were engineered using a ZsGreen cassette driven by the MND promoter. FIG. 15B shows that in-out PCR confirmed site-specific, precise integration of the cassette into the HSCs. FIG. 15C shows that the engineered cells demonstrated proliferative capacity and multi-lineage capacity in methylcellulose colony-forming cell assays.

FIGS. 16A and 16B. FIG. 16A shows that the precision engineering used to gene edit T cells to make the NeoTCR Producs can be applied to natural killer cells (NK cells). Specifically, NK cells were engineered using a ZsGreen cassette driven by the MND promoter and in-out PCR confirmed site-specific, precise integration of the cassette into the NK cells. FIG. 16B shows that high levels of ZsGreen expression were observed in a significant fraction of the CD3−/CD5−/CD56+ engineered cell population 11 days post-transfection.

FIG. 17. FIG. 17 shows a plot of phenotyping experiments on T cells that were gene edited to express the neo12 NeoTCR as described herein. N=5 for each data point.

### DETAILED DESCRIPTION

1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

It is understood that aspects and embodiments of the invention described herein include “comprising,” “consisting,” and “consisting essentially of” aspects and embodiments. The terms “comprises” and “comprising” are intended to have the broad meaning ascribed to them in U.S. Patent Law and can mean “includes”, “including” and the like.

The terms “Cancer” and “Tumor” are used interchangeably herein. As used herein, the terms “Cancer” or “Tumor” refer to all neoplastic cell growth and proliferation, whether malignant or benign, and all pre-cancerous and cancerous cells and tissues. The terms are further used to refer to or describe the physiological condition in mammals that is typically characterized by unregulated cell growth/proliferation. Cancer can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Cancer includes cancers, such as sarcomas, carcinomas, or plasmacytomas (malignant tumor of the plasma cells). Examples of cancer include, but are not limited to, those described herein. The terms “Cancer” or “Tumor” and “Proliferative Disorder” are not mutually exclusive as used herein.

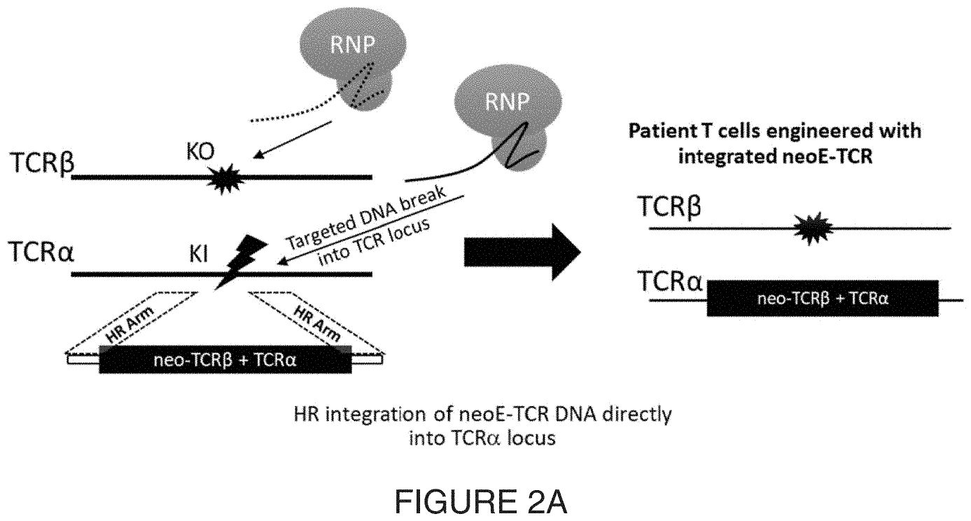

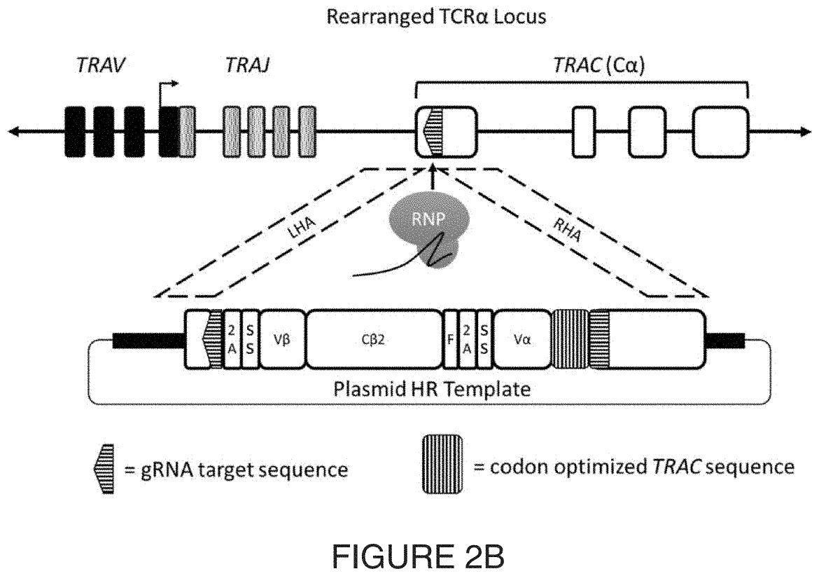

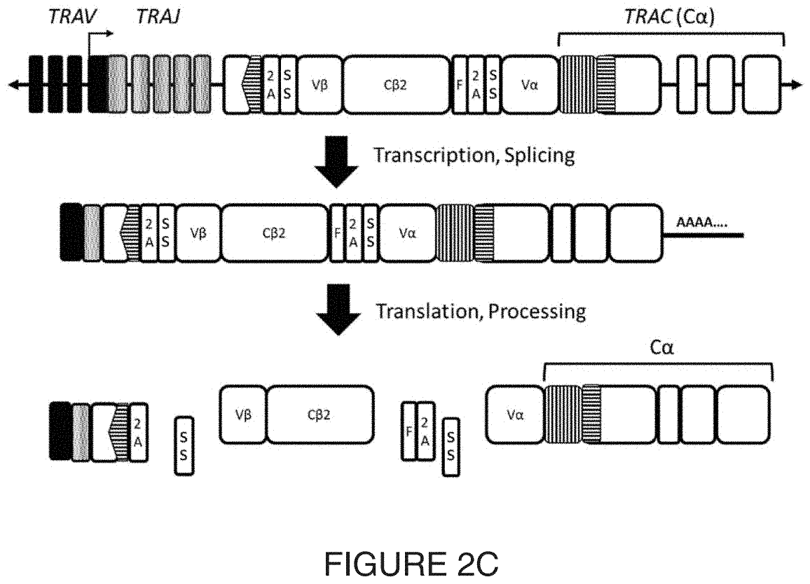

“Cell Product” as used herein means a gene edited cell therapy wherein one or more 2A peptides are used in the gene editing process. In certain embodiments, the Cell Product is made through the insertion of DNA wherein the gene of interest is inserted between two 2A sequences (see, e.g., FIG. 2A). In certain embodiments, the DNA is linear or circular (e.g., plasmid DNA). In certain embodiments, the Cell Product is made through the insertion of DNA wherein the gene of interest is flanked on one side by a 2A peptide. In certain embodiments, when there are more than one 2A peptide sequence, such sequences are the same 2A peptides (e.g., two P2A sequences, two T2A sequences, two E2A sequences, or 2 F2A sequences). In certain embodiments, when there are more than one 2A peptide sequence, such sequences are different 2A peptides (e.g., but not limited to, one T2A and one P2A). In certain embodiments, Cell Products are made using viral gene editing methods. In certain embodiments, Cell Products are made using non-viral gene editing methods. Cell Products include but are not limited to T cell products and NK cell products. Cell Products can also include any other naturally occurring cell that can be edited using a 2A peptide as part of the gene editing process. Cell Products can be used, for example, for the treatment of autoimmune diseases, neurological diseases and injuries (including but not limited to Alzheimer's disease, Parkinson's disease, spinal cord and nerve injuries and/or damage), cancer, infectious diseases, joint disease (including but not limited to rebuilding damaged cartilage in joints), improving the immune system, cardiovascular disease and abnormalities, aging, immune deficiencies (including but not limited to multiple sclerosis and amyotrophic lateral sclerosis), allergies, and genetic disorders.

Cell Products include NeoTCR Products.

“Dextramer” as used herein means a multimerized neoepitope-HLA complex that specifically binds to its cognate NeoTCR.

“NeoTCR” and “NeoE TCR” as used herein mean a neoepitope-specific T cell receptor that is introduced into a T cell, e.g., by gene editing methods.

“NeoTCR cells” as used herein means one or more cells precision engineered to express one or more NeoTCRs. In certain embodiments, the cells are T cells. In certain embodiments, the T cells are CD8+ and/or CD4+ T cells. In certain embodiments, the CD8+ and/or CD4+ T cells are autologous cells from the patient for whom a NeoTCR Product will be administered. The terms “NeoTCR cells” and “NeoTCR-P1 T cells” and “NeoTCR-P1 cells” are used interchangeably herein.

“NeoTCR Product” as used herein means a pharmaceutical formulation comprising one or more NeoTCR cells. NeoTCR Product consists of autologous precision genome-engineered CD8+ and CD4+ T cells. Using a targeted DNA-mediated non-viral precision genome engineering approach, expression of the endogenous TCR is eliminated and replaced by a patient-specific NeoTCR isolated from peripheral CD8+ T cells targeting the tumor-exclusive neoepitope. In certain embodiments, the resulting engineered CD8+ or CD4+ T cells express NeoTCRs on their surface of native sequence, native expression levels, and native TCR function. The sequences of the NeoTCR external binding domain and cytoplasmic signaling domains are unmodified from the TCR isolated from native CD8+ T cells. Regulation of the NeoTCR gene expression is driven by the native endogenous TCR promoter positioned upstream of where the NeoTCR gene cassette is integrated into the genome. Through this approach, native levels of NeoTCR expression are observed in unstimulated and antigen-activated T cell states.

The NeoTCR Product manufactured for each patient represents a defined dose of autologous CD8+ and/or CD4+ T cells that are precision genome engineered to express a single neoE-specific TCR cloned from neoE-specific CD8+ T cells individually isolated from the peripheral blood of that same patient.

NeoTCR Products are non-limiting examples of Cell Products.

“Pharmaceutical Formulation” refers to a preparation which is in such form as to permit the biological activity of an active ingredient contained therein to be effective, and which contains no additional components which are unacceptably toxic to a subject to which the formulation would be administered. For clarity, DMSO at quantities used in a NeoTCR Product are not considered unacceptably toxic.

A “subject,” “patient,” or an “individual” for purposes of treatment refers to any animal classified as a mammal, including humans, domestic and farm animals, and zoo, sports, or pet animals, such as dogs, horses, cats, cows, etc. Preferably, the mammal is human.

“TCR” as used herein means T cell receptor.

“Treat,” “Treatment,” and “treating” are used interchangeably and as used herein mean obtaining beneficial or desired results including clinical results. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the NeoTCR Product of the disclosure is used to delay the development of a proliferative disorder (e.g., cancer) or to slow the progression of such disease.

“2A” and “2A peptide” are used interchangeably herein and mean a class of 18-22 amino acid long, viral, self-cleaving peptides that are able to mediate cleavage of peptides during translation in eukaryotic cells.

Four well-known members of the 2A peptide class are T2A, P2A, E2A, and F2A. The T2A peptide was first identified in the Thosea asigna virus 2A. The P2A peptide was first identified in the porcine teschovirus-1 2A. The E2A peptide was first identified in the equine rhinitis A virus. The F2A peptide was first identified in the foot-and-mouth disease virus.

The self-cleaving mechanism of the 2A peptides is a result of ribosome skipping the formation of a glycyl-prolyl peptide bond at the C-terminus of the 2A. Specifically, the 2A peptides have a C-terminal conserved sequence that is necessary for the creation of steric hindrance and ribosome skipping. The ribosome skipping can result in one of three options: 1) successful skipping and recommencement of translation resulting in two cleaved proteins (the upstream of the 2A protein which is attached to the complete 2A peptide except for the C-terminal proline and the downstream of the 2A protein which is attached to one proline at the N-terminal; 2) successful skipping but ribosome fall-off that results in discontinued translation and only the protein upstream of the 2A; or 3) unsuccessful skipping and continued translation (i.e., a fusion protein).

“Young” or “Younger” or “Young T cell” as it relates to T cells means memory stem cells (T_{MSC}) and central memory cells (T_{CM}). These cells have T cell proliferation upon specific activation and are competent for multiple cell divisions. They also have the ability to engraft after re-infusion, to rapidly differentiate into effector T cells upon exposure to their cognate antigen and target and kill tumor cells, as well as to persist for ongoing cancer surveillance and control.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen-recognizing receptor or capable of suppressing an immune response via receptor-ligand binding.

As used herein, the terms “neoantigen”, “neoepitope” or “neoE” refer to a newly formed antigenic determinant that arise, e.g., from a somatic mutation(s) and is recognized as “non-self.” A mutation giving rise to a “neoantigen”, “neoepitope” or “neoE” can include a frameshift or non-frameshift indel, missense or nonsense substitution, splice site alteration (e.g., alternatively spliced transcripts), genomic rearrangement or gene fusion, any genomic or expression alterations, or any post-translational modifications.

2. NeoTCR Product

In some embodiments, using the neoTCR isolation technology described in PCT/US2020/17887 and PCT/US2019/025415, which are incorporated herein in their entireties. NeoTCRs are cloned in autologous CD8+ and CD4+ T cells from the same patient with cancer by precision genome engineered (using a DNA-mediated (non-viral) method as described in FIGS. 2A-2C) to express the neoTCR. In otherwords, the NeoTCRs that are tumor specific are identified in cancer patients, such NeoTCRs are then cloned, and then the cloned NeoTCRs are inserted into the cancer patient's own T cells. NeoTCR expressing T cells are then expanded in a manner that preserves a “young” T cell phenotypes, resulting in a NeoTCR-P1 product (i.e., a NeoTCR Product) in which the majority of the T cells exhibit T memory stem cell and T central memory phenotypes.

These ‘young’ or ‘younger’ or less-differentiated T cell phenotypes are described to confer improved engraftment potential and prolonged persistence post-infusion. Thus, the administration of NeoTCR Product, consisting significantly of ‘young’ T cell phenotypes, has the potential to benefit patients with cancer, through improved engraftment potential, prolonged persistence post-infusion, and rapid differentiation into effector T cells to eradicate tumor cells throughout the body.

Ex vivo mechanism-of-action studies were also performed with NeoTCR Product manufactured with T cells from patients with cancer. Comparable gene editing efficiencies and functional activities, as measured by antigen-specificity of T cell killing activity, proliferation, and cytokine production, were observed demonstrating that the manufacturing process described herein is successful in generating product with T cells from patients with cancer as starting material.

The NeoTCR Product manufacturing process involves electroporation of dual ribonucleoprotein species of CRISPR-Cas9 nucleases bound to guide RNA sequences, with each species targeting the genomic TCRα and the genomic TCRβ loci. The specificity of targeting Cas9 nucleases to each genomic locus has been previously described in the literature as being highly specific. Comprehensive testing of the NeoTCR Product was performed in vitro and in silico analyses to survey possible off-target genomic cleavage sites, using COSMID and GUIDE-seq, respectively. Multiple NeoTCR Product or comparable cell products from healthy donors were assessed for cleavage of the candidate off-target sites by deep sequencing, supporting the published evidence that the selected nucleases are highly specific.

Further aspects of the precision genome engineering process have been assessed for safety. No evidence of genomic instability following precision genome engineering was found in assessing multiple NeoTCR Products by targeted locus amplification (TLA) or standard FISH cytogenetics. No off-target integration anywhere into the genome of the NeoTCR sequence was detected. No evidence of residual Cas9 was found in the cell product.

The comprehensive assessment of the NeoTCR Product and precision genome engineering process indicates that the NeoTCR Product will be well tolerated following infusion back to the patient.

The genome engineering approach described herein enables highly efficient generation of bespoke NeoTCR T cells (i.e., NeoTCR Products) for personalized adoptive cell therapy for patients with solid and liquid tumors. Furthermore, the engineering method is not restricted to the use in T cells and has also been applied successfully to other primary cell types, including natural killer and hematopoietic stem cells.

3. Pharmaceutical Formulations

Pharmaceutical formulations of the NeoTCR Product are prepared by combining the NeoTCR cells in a solution that can preserve the ‘young’ phenotype of the cells in a cryopreserved state. Table 1 provides an example of one such pharmaceutical formulation. Alternatively, pharmaceutical formulations of the NeoTCR Product can be prepared by combining the NeoTCR cells in a solution that can preserve the ‘young’ phenotype of the cells without the need to freeze or cryopreserve the product (i.e., the NeoTCR Product is maintained in an aqueous solution or as a non-frozen/cryopreserved cell pellet).

Additional pharmaceutically acceptable carriers, buffers, stabilizers, and/or preservatives can also be added to the cryopreservation solution or the aqueous storage solution (if the NeoTCR Product is not cryopreserved). Any cryopreservation agent and/or media can be used to cryopreserve the

NeoTCR Product, including but not limited to CryoStor, CryoStor CS5, CELLBANKER, and custom cryopreservation medias that optionally include DMSO.

4. Gene-Editing Methods

In certain embodiments, the present disclosure involves, in part, methods of engineering human cells, e.g., engineered T cells or engineered human stem cells. In certain embodiments, such engineering involves genome editing. For example, but not by way of limitation, such genome editing can be accomplished with nucleases targeting one or more endogenous loci, e.g., TCR alpha (TCRa) locus and TCR beta (TCRβ) locus. In certain embodiments, the nucleases can generate single-stranded DNA nicks or double-stranded DNA breaks in an endogenous target sequence. In certain embodiments, the nuclease can target coding or non-coding portions of the genome, e.g., exons, introns. In certain embodiments, the nucleases contemplated herein comprise homing endonuclease, meganuclease, megaTAL nuclease, transcription activator-like effector nuclease (TALEN), zinc-finger nuclease (ZFN), and clustered regularly interspaced short palindromic repeats (CRISPR)/Cas nuclease. In certain embodiments, the nucleases can themselves be engineered, e.g., via the introduction of amino acid substitutions and/or deletions, to increase the efficiency of the cutting activity.

In certain embodiments, a CRISPR/Cas nuclease system is used to engineer human cells. In certain embodiments, the CRISPR/Cas nuclease system comprises a Cas nuclease and one or more RNAs that recruit the Cas nuclease to the endogenous target sequence, e.g., single guide RNA. In certain embodiments, the Cas nuclease and the RNA are introduced in the cell separately, e.g. using different vectors or compositions, or together, e.g., in a polycistronic construct or a single protein-RNA complex. In certain embodiments, the Cas nuclease is Cas9 or Cas12a. In certain embodiments, the Cas9 polypeptide is obtained from a bacterial species including, without limitation, Streptococcus pyogenes or Neisseria menengitidis. Additional example of CRISPR/Cas systems are known in the art. See Adli, Mazhar. “The CRISPR tool kit for genome editing and beyond.” Nature communications vol. 9,1 1911 (2018), herein incorporated by reference for all that it teaches.

In certain embodiments, genome editing occurs at one or more genome loci that regulate immunological responses. In certain embodiments, the loci include, without limitation, TCR alpha (TCRα) locus, TCR beta (TCRβ) locus, TCR gamma (TCRγ), TCR delta (TCRδ).

In certain embodiments, genome editing is performed by using non-viral delivery systems. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

5. Homology Recombination Templates

In certain embodiments, the present disclosure provides genome editing of a cell by introducing and recombining a homologous recombination (HR) template nucleic acid sequence into an endogenous locus of a cell. In certain embodiments, the HR template nucleic acid sequence is linear. In certain embodiments, the HR template nucleic acid sequence is circular. In certain embodiments, the circular HR template can be a plasmid, minicircle, or nanoplasmid. In certain embodiments, the HR template nucleic acid sequence comprises a first and a second homology arms. In certain embodiments, the homology arms can be of about 300 bases to about 2,000 bases. For example, each homology arm can be 1,000 bases. In certain embodiments, the homology arms can be homologous to a first and second endogenous sequences of the cell. In certain embodiments, the endogenous locus is a TCR locus. For example, the first and second endogenous sequences are within a TCR alpha locus or a TCR beta locus. In certain embodiments, the HR template comprises a TCR gene sequences. In non-limiting embodiments, the TCR gene sequence is a patient specific TCR gene sequence. In non-limiting embodiments, the TCR gene sequence is tumor-specific. In non-limiting embodiments, the TCR gene sequence can be identified and obtained using the methods described in PCT/US2020/017887, the content of which is herein incorporated by reference. In certain embodiments, the HR template comprises a TCR alpha gene sequence and a TCR beta gene sequence.

In certain embodiments, the HR template is a polycistronic polynucleotide. In certain embodiments, the HR template comprises sequences encoding for flexible polypeptide sequences (e.g., Gly-Ser-Gly sequence). In certain embodiments, the HR template comprises sequences encoding an internal ribosome entry site (IRES). In certain embodiments, the HR template comprises a 2A peptide (e.g., P2A, T2A, E2A, and F2A). Additional information on the HR template nucleic acids and methods of modifying a cell thereof can be found in International Patent Application no. PCT/US2018/058230, the content of which is herein incorporated by reference.

6. Methods of Producing Engineered Young T Cells

In certain embodiments, the present disclosure relates, in part, on the production of engineered “young” T cells. In certain embodiments, the present disclosure comprises methods for producing antigen-specific cells, e.g., T cells, ex vivo, comprising activating, engineering, and expanding antigen-specific cells originally obtained from a subject or isolated from such sample.

In certain embodiments, the methods for activating cells comprise the steps of activating the TCR/CD3 complex. For example, without limitation, the T cells can be incubated and/or cultured with CD3 agonists, CD28 agonists, or a combination thereof In certain embodiments activated antigen-specific cells are engineered as described herein, e.g., Sections 4 and 5, above, and the Examples, below.

In certain embodiments, engineered activated antigen-specific cells, e.g., engineered activated T cells, can be expanded by culturing the engineered activated antigen-specific cells, e.g., T cells, with cytokines, chemokine, soluble peptides, or combination thereof. In certain embodiments, the engineered activated antigen-specific cells, e.g., engineered activated T cells, can be cultured with one or more cytokines. In certain embodiments, the cytokines can be IL2, IL7, IL15, or combinations thereof. For example, engineered activated antigen-specific cells, e.g., engineered activated T cells, can be cultured with IL7 and IL15. In certain embodiments, the cytokine used in connection with the engineered activated antigen-specific cell, e.g., engineered activated T cell, culture can be present at a concentration from about 1 pg/ml to about 1 g/ml, from about 1 ng/ml to about 1 g/ml, from about 1 μg/ml to about 1 g/ml, or from about 1 mg/ml to about 1 g/ml, and any values in between.

7. Articles of Manufacture

The NeoTCR Product can be used in combination with articles of manufacture. Such articles of manufacture can be useful for the prevention or treatment of proliferative disorders (e.g., cancer). Examples of articles of manufacture include but are not limited to containers (e.g., infusion bags, bottles, storage containers, flasks, vials, syringes, tubes, and IV solution bags) and a label or package insert on or associated with the container. The containers may be made of any material that is acceptable for the storage and preservation of the NeoTCR cells in the ‘young’ state within the NeoTCR Product. In certain embodiments, the container may be an intravenous solution bag or a vial having a stopper pierceable by a hypodermic injection needle. For example, the container may be a CryoMACS freezing bag. The label or package insert indicates that the NeoTCR Product is used for treating the condition of choice and the patient of origin. The patient is identified on the container of the NeoTCR Product because the NeoTCR Product is made from autologous cells and engineered as a patient-specific and individualized treatment.

The article of manufacture may comprise: 1) a first container with a NeoTCR Product contained therein.

The article of manufacture may comprise: 1) a first container with a NeoTCR Product contained therein; and 2) a second container with the same NeoTCR Product as the first container contained therein. Optionally, additional containers with the same NeoTCR Product as the first and second containers may be prepared and made. Optionally, additional containers containing a composition comprising a different cytotoxic or otherwise therapeutic agent may also be combined with the containers described above.

The article of manufacture may comprise: 1) a first container with a NeoTCR Product contained therein; and 2) a second container with a composition contained therein, wherein the composition comprises a further cytotoxic or otherwise therapeutic agent.

The article of manufacture may comprise: 1) a first container with two NeoTCR Products contained therein; and 2) a second container with a composition contained therein, wherein the composition comprises a further cytotoxic or otherwise therapeutic agent.

The article of manufacture may comprise: 1) a first container with a NeoTCR Product contained therein; 2) a second container with a second NeoTCR Product contained therein; and 3) optionally a third container with a composition contained therein, wherein the composition comprises a further cytotoxic or otherwise therapeutic agent. In certain embodiments, the first and second NeoTCR Products are different NeoTCR Products. In certain embodiments, the first and second NeoTCR Products are the same NeoTCR Products.

The article of manufacture may comprise: 1) a first container with three NeoTCR Products contained therein; and 2) optionally a second container with a composition contained therein, wherein the composition comprises a further cytotoxic or otherwise therapeutic agent.

The article of manufacture may comprise: 1) a first container with a NeoTCR Product contained therein; 2) a second container with a second NeoTCR Product contained therein; 3) a third container with a third NeoTCR Product contained therein; and 4) optionally a fourth container with a composition contained therein, wherein the composition comprises a further cytotoxic or otherwise therapeutic agent. In certain embodiments, the first, second, and third NeoTCR Products are different NeoTCR Products. In certain embodiments, the first, second, and third NeoTCR Products are the same NeoTCR Products. In certain embodiments, two of the first, second, and third NeoTCR Products are the same NeoTCR Products.

The article of manufacture may comprise: 1) a first container with four NeoTCR Products contained therein; and 2) optionally a second container with a composition contained therein, wherein the composition comprises a further cytotoxic or otherwise therapeutic agent.

The article of manufacture may comprise: 1) a first container with a NeoTCR Product contained therein; 2) a second container with a second NeoTCR Product contained therein; 3) a third container with a third NeoTCR Product contained therein; 4) a fourth container with a fourth NeoTCR Product contained therein; and 5) optionally a fifth container with a composition contained therein, wherein the composition comprises a further cytotoxic or otherwise therapeutic agent. In certain embodiments, the first, second, third, and fourth NeoTCR Products are different NeoTCR Products. In certain embodiments, the first, second, third, and fourth NeoTCR Products are the same NeoTCR Products. In certain embodiments, two of the first, second, third, and fourth NeoTCR Products are the same NeoTCR Products. In certain embodiments, three of the first, second, third, and fourth NeoTCR Products are the same NeoTCR Products.

The article of manufacture may comprise: 1) a first container with five or more NeoTCR Products contained therein; and 2) optionally a second container with a composition contained therein, wherein the composition comprises a further cytotoxic or otherwise therapeutic agent.

The article of manufacture may comprise: 1) a first container with a NeoTCR Product contained therein; 2) a second container with a second NeoTCR Product contained therein; 3) a third container with a third NeoTCR Product contained therein; 4) a fourth container with a fourth NeoTCR Product contained therein; 5) a fifth container with a fifth NeoTCR Product contained therein; 6) optionally a sixth or more additional containers with a sixth or more NeoTCR Product contained therein; and 7) optionally an additional container with a composition contained therein, wherein the composition comprises a further cytotoxic or otherwise therapeutic agent. In certain embodiments, the all of the containers of NeoTCR Products are different NeoTCR Products. In certain embodiments, all of the containers of NeoTCR Products are the same NeoTCR Products. In certain embodiments, there can be any combination of same or different NeoTCR Products in the five or more containers based on the availability of detectable NeoTCRs in a patient's tumor sample(s), the need and/or desire to have multiple NeoTCR Products for the patient, and the availability of any one NeoTCR Product that may require or benefit from one or more container.

Furthermore, any container of NeoTCR Product described herein can be split into two, three, or four separate containers for multiple time points of administration and/or based on the appropriate dose for the patient.

In certain embodiments, the NeoTCR Products are provided in a kit. The kit can, by means of non-limiting examples, contain package insert(s), labels, instructions for using the NeoTCR Product(s), syringes, disposal instructions, administration instructions, tubing, needles, and anything else a clinician would need in order to properly administer the NeoTCR Product(s).

8. Therapeutic Composition and Method of Manufacturing

As described herein, plasmid DNA-mediated (non-viral) precision genome engineering process for Good Manufacturing Practice (GMP) manufacturing of NeoTCR Product was developed. Targeted integration of the patient-specific neoTCR is accomplished by electroporating CRISPR endonuclease ribonucleoproteins (RNPs) together with the personalized neoTCR gene cassette, encoded by the plasmid DNA.

The NeoTCR Product was formulated into a drug product using the clinical manufacturing process. Under this process, the NeoTCR Product is cryopreserved in CryoMACS Freezing Bags. One or more bags may be shipped to the site for each patient depending on patient needs. The product is composed of apheresis-derived, patient-autologous, CD8 and CD4 T cells that have been precision genome engineered to express one or more autologous neoTCRs targeting a neoepitope complexed to one of the endogenous HLA receptors presented exclusively on the surface of that patient's tumor cells.

The final product contains 5% dimethyl sulfoxide (DMSO), human serum albumin and Plasma-Lyte. The final cell product contains the list of components provided in Table 1.

TABLE 1
Composition of the NeoTCR Product

| Component | Specification/Grade |
|---|---|
| Total nucleated NeoTCR cells | cGMP manufactured |
| Plasma-Lyte A | USP |
| Human Serum Albumin in | USP |
| 0.02-0.08 M sodium | |
| caprylate and sodium tryptophanate | |
| CryoStor CS10 | cGMP manufactured |
| | with USP grade materials |

9. Pharmacodynamics

The pharmacodynamic studies described herein recapitulate antigen-specific T cell proliferation, cytokine production, and target T cell killing activity using ex vivo assays of precision genome engineered human neoTCR-T cells (i.e, the NeoTCR Product).

Pharmacodynamic studies supporting the mechanism of action of the NeoTCR Product have been performed with NeoTCR cells. The reagents include antibodies used for T cell selection, reagents for precision genome engineering, media, and cytokines. The starting material can be selected from either leukopaks or blood draws. Expansion of the T cells can occur in vessels suitable for T cell survival and expansion such as a G-Rex vessel or a CentriCult vessel. Additional methods of cell expansion can take place in T flasks, culture bags, closed system bioreactors (non-limiting examples include the Xuri system (General Electric), the Ambr system (Sartorius), the Quantum system (Terumo CVT), and the Cocoon system (Lonza), cell stacks that are optionally optimized for non-adherent cells, and cell factories that are optionally optimized for non-adherent cells.

10. Methods of Treatment

The presently disclosed subject matter provides methods for inducing and/or increasing an immune response in a subject in need thereof. The presently disclosed cells and compositions comprising thereof can be used for treating and/or preventing a cancer in a subject. The presently disclosed cells and compositions comprising thereof can be used for prolonging the survival of a subject suffering from a cancer. The presently disclosed cells and compositions comprising thereof can also be used for treating and/or preventing a cancer in a subject. The presently disclosed cells and compositions comprising thereof can also be used for reducing tumor burden in a subject. Such methods comprise administering the presently disclosed cells in an amount effective or a composition (e.g., a pharmaceutical composition) comprising thereof to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

For adoptive immunotherapy using the young T cells disclosed herein, cell doses in the range of about 10⁶-10¹¹ (e.g., about 10⁹) are typically infused. Upon administration of the presently disclosed cells into the host and subsequent differentiation, younger T cells are induced that are specifically directed against the specific antigen. The presently disclosed cells can be administered by any method known in the art including, but not limited to, intravenous, subcutaneous, intranodal, intratumoral, intrathecal, intrapleural, intraperitoneal, intra-medullary and directly to the thymus.

The presently disclosed subject matter provides methods for treating and/or preventing cancer in a subject. In certain embodiments, the method comprises administering an effective amount of the presently disclosed cells or a composition comprising thereof to a subject having cancer.

Non-limiting examples of cancer include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangloblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasia is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed young T cells and compositions comprising thereof can be used for treating and/or preventing blood cancers (e.g., leukemias, lymphomas, and myelomas) or ovarian cancer, which are not amenable to conventional therapeutic interventions.

In certain embodiments, the neoplasia is a solid cancer or a solid tumor. In certain embodiments, the solid tumor or solid cancer is selected from the group consisting of glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, lung adenocarcinoma, stomach adenocarcinoma, cervical and endocervical cancer, kidney clear cell carcinoma, testicular germ cell tumors, and aggressive B-cell lymphomas.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

As a consequence of the expression of TCR that binds to a patient-derived tumor antigen or neoantigen, adoptively transferred young T cells are endowed with augmented and selective cytolytic activity at the tumor site and their response does not undergo to exhaustion. Furthermore, subsequent to their localization to tumor and their proliferation, the young T cells turn the tumor site into a highly conductive environment for a wide range of immune cells involved in the physiological anti-tumor response (tumor infiltrating lymphocytes, NK-, NKT-cells, dendritic cells, and macrophages).

11. Exemplary Embodiments

A. In certain non-limiting embodiments, the presently disclosed subject matter provides for a method of producing a population of modified young T cells, comprising: a) introducing into a T cell a homologous recombination (HR) template nucleic acid sequence comprising: first and second homology arms homologous to first and second target nucleic acid sequences; a TCR gene sequence positioned between the first and second homology arms; b) recombining the HR template nucleic acid into an endogenous locus of the cell comprising the first and second endogenous sequences homologous to the first and second homology arms of the HR template nucleic acid; and c) culturing the T cell to produce a population of young T cells.

A1. The foregoing method of A, wherein the HR template comprises a first 2A-coding sequence positioned upstream of the TCR gene sequence and a second 2A-coding sequence positioned downstream of the TCR gene sequence, wherein the first and second 2A-coding sequences code for the same amino acid sequence that are codon-diverged relative to each other.

A2. The foregoing method of A or A1, wherein the 2A-coding sequence is a P2A-coding sequence.

A3. The foregoing method of A1 or A2, wherein a sequence coding for the amino acid sequence Gly Ser Gly is positioned immediately upstream of the 2A-coding sequences.

A4. The foregoing method of any one of A1-A3, wherein the HR template comprises a sequence coding for a Furin cleavage site positioned upstream of the second 2A-coding sequence.

A5. The foregoing method of any one of A-A4, wherein the first and second homology arms are each from about 300 bases to about 2,000 bases in length.

A6. The foregoing method of any one of A-A5, wherein the first and second homology arms are each from about 600 bases to about 2,000 bases in length.

A7. The foregoing method of any one of A1-A6, wherein the HR template further comprises a signal sequence positioned between the first 2A-coding sequence and the TCR gene sequence.

A8. The foregoing method of any one of A1-A7, wherein the HR template comprises a second TCR sequence positioned between the second 2A-coding sequence and the second homology arm.

A9. The foregoing method of A8, wherein the HR template comprises: a first signal sequence positioned between the first 2A-coding sequence and the first TCR gene sequence; and a second signal sequence positioned between the second 2A-coding sequence and the second TCR gene sequence; wherein the first and the second signal sequences encode for the same amino acid sequence and are codon diverged relative to each other.

A10. The foregoing method of A7 or A9, wherein the signal sequence is a human growth hormone signal sequence.

A11. The foregoing method of any one of A-A 10, wherein the HR template is non-viral.

A12. The foregoing method of any one of A-A11, wherein the HR template is a circular DNA.

A13. The foregoing method of any one of A-A12, wherein the HR template is a linear DNA.

A14. The foregoing method of any one of A-A13, wherein the T cell is a patient-derived cell.

A15. The foregoing method of any one of A-A14, wherein the endogenous locus is within an endogenous TCR gene.

A16. The foregoing method of any one of A-A15, wherein the TCR gene sequence encodes for a TCR that recognizes a tumor antigen.

A17. The foregoing method of A16, wherein the tumor antigen is a neoantigen.

A18. The foregoing method of A16, wherein the tumor antigen is a patient specific neoantigen.

A19. The foregoing method of any one of A-A18, wherein the TCR gene sequence is a patient specific TCR gene sequence.

A20. The foregoing method of any one of A-A19, wherein said recombining comprises: cleavage of the endogenous locus by a nuclease; and recombination of the HR template nucleic acid sequence into the endogenous locus by homology directed repair.

A21. The foregoing method of A20, wherein the nuclease is a Clustered Regularly Interspaced Short Palindromic Repeats (CRISPR) family nuclease or derivative thereof.

A22. The foregoing method of A21, further comprising an sgRNA.

A23. The foregoing method of any one of A-A22, wherein the introducing occurs via electroporation.

A24. The foregoing method of any one of A-A23, wherein the culturing is conducted in the presence of at least one cytokine.

A25. The foregoing method of A24, wherein the culturing is conducted in the presence of IL2, IL7, IL15, or any combination thereof.

A26. The foregoing method of A24, wherein the culturing is conducted in the presence of IL7 and IL15.

A27. The foregoing method of any one of A-A26, wherein the population of young T cells comprises cells that are CD45RA+, CD62L+, CD28+, CD95−, CCR7+, and CD27+.

A28. The foregoing method of any one of A-A26, wherein the population of young T cells comprises cells that are CD45RA+, CD62L+, CD28+, CD95+, CD27+, CCR7+.

A29. The foregoing method of any one of A-A26, wherein the population of young T cells comprises cells that are CD45RO+, CD62L+, CD28+, CD95+, CCR7+, CD27+, CD127+.

A30. The foregoing method of any one of A-A29, wherein the population of young T cells maintains its killing activity for at least about 14 days-60 days.

A31. The foregoing method of any one of A-A29, wherein the population of young T cells maintains its killing activity for at least about 61 days-120 days.

A32. The foregoing method of any one of A-A29, wherein the population of young T cells maintains its killing activity for more than 120 days.

B. In certain non-limiting embodiments, the presently disclosed subject matter provides for a population of young T cells obtained by the method of any one of A-A32.

C. In certain non-limiting embodiments, the presently disclosed subject matter provides for a pharmaceutical composition comprising the population of young T cells of B.

C1. The foregoing pharmaceutical composition of C, wherein the composition is administered to a patient in need thereof for the treatment of cancer, and wherein cells of the composition engraft in the patient as Tmsc or Tcm cells.

D. In certain non-limiting embodiments, the presently disclosed subject matter provides for a method of treating cancer in a subject in need thereof, the method comprising: a) modifying patient-derived T cells by introducing a homologous recombination (HR) template into the T cell, wherein the HR template comprises: first and second homology arms homologous to first and second target nucleic acid sequences; a TCR gene sequence positioned between the first and second homology arms; b) recombining the polynucleotide into an endogenous locus of the T cell; c) culturing the modified T cell to produce a population of young T cells; and d) administering a therapeutically effective amount of the population of modified young T cells to the human patient to thereby treat the cancer.

D1. The foregoing method of D, wherein prior to administering a therapeutically effective amount of modified young T cells, a non-myeloablative lymphodepletion regimen is administered to the subject.

D2. The foregoing method of D or D1, wherein the cancer is a solid tumor.

D3. The foregoing method of D or D1, wherein the cancer is a liquid tumor.

D4. The foregoing method of D2, wherein the solid tumor selected from the group consisting of melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinoma, renal cell cancers, testicular cancer, sarcomas, and colorectal cancer.

D5. The foregoing method of D3, wherein the liquid tumor is selected from the group consisting of follicular lymphoma, leukemia, and multiple myeloma.

D6. The foregoing method of any one of D-D5, wherein the HR template comprises a first 2A-coding sequence positioned upstream of the TCR gene sequence and a second 2A-coding sequence positioned downstream of the TCR gene sequence, wherein the first and second 2A-coding sequences code for the same amino acid sequence that are codon-diverged relative to each other.

D7. The foregoing method of D6, wherein the 2A-coding sequence is a P2A-coding sequence.

D8. The foregoing method of D6 or D7, wherein a sequence coding for the amino acid sequence Gly Ser Gly is positioned immediately upstream of the 2A-coding sequences.

D9. The foregoing method of any one of D6-D8, wherein the HR template comprises a sequence coding for a Furin cleavage site positioned upstream of the second 2A-coding sequence.

D10. The foregoing method of any one of D-D9, wherein the first and second homology arms are each from about 300 bases to about 2,000 bases in length.

D11. The foregoing method of any one of D-D10, wherein the first and second homology arms are each from about 600 bases to about 2,000 bases in length.

D12. The foregoing method of any one of D-D11, wherein the HR template further comprises a signal sequence positioned between the first 2A-coding sequence and the TCR gene sequence.

D13. The foregoing method of any one of D-D12, wherein the HR template comprises a second TCR sequence positioned between the second 2A-coding sequence and the second homology arm.

D14. The foregoing method of any one of D-D13, wherein the HR template comprises: a first signal sequence positioned between the first 2A-coding sequence and the first TCR gene sequence; and a second signal sequence positioned between the second 2A-coding sequence and the second TCR gene sequence; wherein the first and the second signal sequences encode for the same amino acid sequence and are codon diverged relative to each other.

D15. The foregoing method of any one of D12-D14, wherein the signal sequence is a human growth hormone signal sequence.

D16. The foregoing method of any one of D-D15, wherein the HR template is non-viral.

D17. The foregoing method of any one of D-D16, wherein the HR template is a circular DNA.

D18. The foregoing method of any one of D-D17, wherein the HR template is a linear DNA.

D19. The foregoing method of any one of D-D18, wherein the endogenous locus is within an endogenous TCR gene.

D20. The foregoing method of any one of D-D19, wherein the TCR gene sequence encodes for a TCR that recognizes a tumor antigen.

D21. The foregoing method of D20, wherein the tumor antigen is a neoantigen.

D22. The foregoing method of D20, wherein the tumor antigen is a patient specific neoantigen.

D23. The foregoing method of any one of D-D22, wherein the TCR gene sequence is a patient specific TCR gene sequence.

D24. The foregoing method of any one of D-D23, wherein said recombining comprises: cleavage of the endogenous locus by a nuclease; and recombination of the HR template nucleic acid sequence into the endogenous locus by homology directed repair.

D25. The foregoing method of D24, wherein the nuclease is a Clustered Regularly Interspaced Short Palindromic Repeats (CRISPR) family nuclease or derivative thereof.

D26. The foregoing method of D25, further comprising an sgRNA.

D27. The foregoing method of any one of D-D26, wherein the introducing occurs via electroporation.

D28. The foregoing method of any one of D-D27, wherein the culturing is conducted in the presence of at least one cytokine.

D29. The foregoing method of D28, wherein the culturing is conducted in the presence of IL2, IL7, IL15, or any combination thereof.

D30. The foregoing method of D28, wherein the culturing is conducted in the presence of IL7 and IL15.

D31. The foregoing method of any one of D-D30, wherein the population of young T cells comprises cells that are CD45RA+, CD62L+, CD28+, CD95−, CCR7+, and CD27+.

D32. The foregoing method of any one of D-D30, wherein the population of young T cells comprises cells that are CD45RA+, CD62L+, CD28+, CD95+, CD27+, CCR7+.

D33. The foregoing method of any one of D-D30, wherein the population of young T cells comprises cells that are CD45RO+, CD62L+, CD28+, CD95+, CCR7+, CD27+, CD127+.

D34. The foregoing method of any one of D-D33, wherein the population of young T cell maintains its killing activity for at least about 14 days.

D35. The foregoing method of any one of D-D34, wherein the population of young T cells maintains its killing activity for at least about 60 days, or between 60 days and 120 days, or 121 days and 180 days, or 181 days and 250 days, or 251 days and 365 days, or greater than one year.

D36. The foregoing method of any one of D-D35, wherein the population of young T cells engrafts into the patient following the administration of a therapeutically effective amount of the population of modified young T cells.

D37. The foregoing method of D36, wherein the engrafted cells activate upon neoantigen presentation on a tumor cell, and wherein the engrafted cells kill the tumor cell.

D38. The foregoing method of D37, wherein the engrafted cells can activate and kill a tumor cell for up to 30 days, for 31-60 days, for 61-90 days, for 91-180 days, for 181-250 days, for 251-265 days, or for over 1 year following administration to the patient.

E. In certain non-limiting embodiments, the presently disclosed subject matter provides for a method of modifying a cell, wherein the cell is a natural killer cell or hematopoietic stem cell, the method comprising: a) introducing into the cell a homologous recombination (HR) template nucleic acid sequence comprising: first and second homology arms homologous to first and second target nucleic acid sequences; a TCR gene sequence positioned between the first and second homology arms; b) recombining the HR template nucleic acid into an endogenous locus of the cell comprising the first and second endogenous sequences homologous to the first and second homology arms of the HR template nucleic acid.

E1. The foregoing method of E, wherein the HR template comprises a first 2A-coding sequence positioned upstream of the TCR gene sequence and a second 2A-coding sequence positioned downstream of the TCR gene sequence, wherein the first and second 2A-coding sequences code for the same amino acid sequence that are codon-diverged relative to each other.

E2. The foregoing method of E or El, wherein the 2A-coding sequence is a P2A-coding sequence.

E3. The foregoing method of any one of E-E2, wherein a sequence coding for the amino acid sequence Gly Ser Gly is positioned immediately upstream of the 2A-coding sequences.

E4. The foregoing method of any one of E-E3, wherein the HR template comprises a sequence coding for a Furin cleavage site positioned upstream of the second 2A-coding sequence.

E5. The foregoing method of any one of E-E4, wherein the first and second homology arms are each from about 300 bases to about 2,000 bases in length.

E6. The foregoing method of any one of E-E5, wherein the HR template further comprises a signal sequence positioned between the first 2A-coding sequence and the TCR gene sequence.

E7. The foregoing method of any one of E-E6, wherein the HR template comprises a second TCR sequence positioned between the second 2A-coding sequence and the second homology arm.

E8. The method of any one of E-E7, wherein the HR template comprises: a first signal sequence positioned between the first 2A-coding sequence and the first TCR gene sequence; and a second signal sequence positioned between the second 2A-coding sequence and the second TCR gene sequence; wherein the first and the second signal sequences encode for the same amino acid sequence and are codon diverged relative to each other.

E9. The method of any one of E6-E8, wherein the signal sequence is a human growth hormone signal sequence.

E10. The foregoing method of any one of E-E9, wherein the HR template is non-viral.

E11. The foregoing method of any one of E-E9, wherein the HR template is a circular DNA.

E12. The foregoing method of any one of E-E9, wherein the HR template is a linear DNA.

E13. The foregoing method of any one of E-E12, wherein the cell is a patient-derived cell.

E14. The foregoing method of any one of E-E13, wherein the endogenous locus is within an endogenous TCR gene.

E15. The foregoing method of any one of E-E14, wherein the TCR gene sequence encodes for a TCR that recognizes a tumor antigen.

E16. The foregoing method of E15, wherein the tumor antigen is a neoantigen.

E17. The foregoing method of E15, wherein the tumor antigen is a patient specific neoantigen.

E18. The foregoing method of any one of E-E17, wherein the TCR gene sequence is a patient specific TCR gene sequence.

E19. The foregoing method of any one of E-E18, wherein said recombining comprises: cleavage of the endogenous locus by a nuclease; and recombination of the HR template nucleic acid sequence into the endogenous locus by homology directed repair.

E20. The foregoing method of E19, wherein the nuclease is a Clustered Regularly Interspaced Short Palindromic Repeats (CRISPR) family nuclease or derivative thereof.

E21. The foregoing method of E20, further comprising an sgRNA.

F. In certain non-limiting embodiments, the presently disclosed subject matter provides for a method of any of A-A24, A26-A32, D-D28, D30-D38, E-E21, wherein IL2 is not used in the culturing.

G. In certain non-limiting embodiments, the presently disclosed subject matter provides for a pharmaceutical formulation comprising young T cells made using any of the methods of A-A32, B,

C-C1, D-D38, E-E21, and F, wherein the pharmaceutical formation comprises at least 20% Tmsc and Tcm collectively, at least 25% Tmsc and Tcm collectively, at least 30% Tmsc and Tcm collectively, at least 35% Tmsc and Tcm collectively, at least 40% Tmsc and Tcm collectively, at least 45% Tmsc and Tcm collectively, at least 50% Tmsc and Tcm collectively, at least 55% Tmsc and Tcm collectively, at least 60% Tmsc and Tcm collectively or more than 61% Tmsc and Tcm collectively.

G1. The foregoing pharmaceutical formulation of G, wherein the final formulation is cryopreserved in 46% Plasma-Lyte A, 1% HSA (w/v), and 50% CryoStor CS10.As a consequence of the expression of TCR that binds to a patient-derived tumor antigen or neoantigen, adoptively transferred young T cells are endowed with augmented and selective cytolytic activity at the tumor site and their response does not undergo to exhaustion. Furthermore, subsequent to their localization to tumor and their proliferation, the young T cells turn the tumor site into a highly conductive environment for a wide range of immune cells

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T.E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3rd Ed. (Plenum Press) Vols A and B(1992).

Provided herein are examples of engineering T cells to express a NeoTCR to create a personalized adoptive T cell therapy (i.e., a NeoTCR Product) which is composed of apheresis-derived, patient-autologous, CD8 and CD4 T cells that have been precision genome engineered to express an autologous T cell receptor targeting a neoepitope presented exclusively on the surface of the patient's tumor cells (neoTCR), wherein the NeoTCR Product comprises T cells with a young phenotype.

### Example 1

### Target Integration

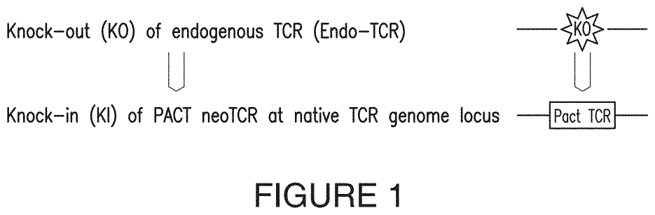

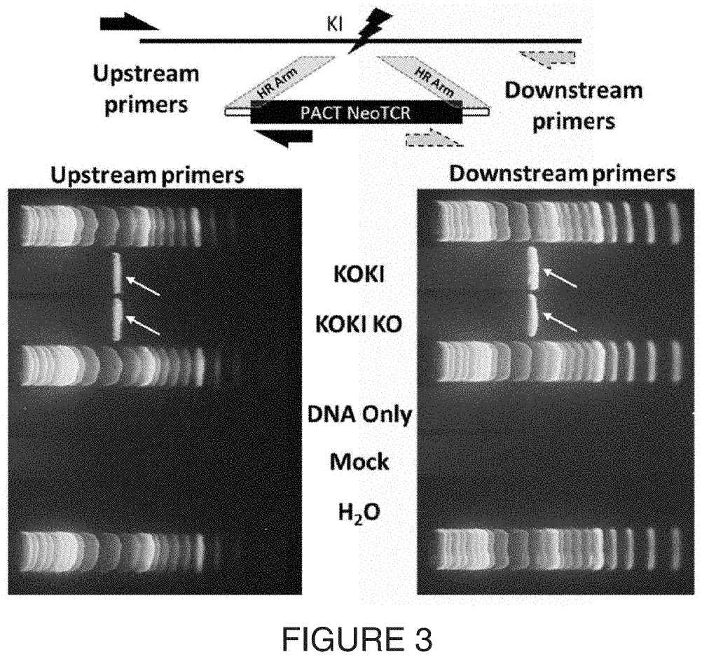

Neoepitope-specific TCRs identified by the imPACT Isolation Technology described in PCT/US2020/17887 (which is herein incorporated by reference in its entirety) were used to generate homologous recombination (HR) DNA templates. These HR templates were transfected into primary human T cells in tandem with site-specific nucleases (see FIG. 1, FIGS. 2A-2C, and FIG. 3).

The single-step non-viral precision genome engineering resulted in the seamless replacement of the endogenous TCR with the patient's neoepitope-specific TCR, expressed by the endogenous promoter. The TCR expressed on the surface is entirely native in sequence.

The precision of neoTCR-T cell genome engineering was evaluated by Targeted Locus Amplification (TLA) for off-target integration hot spots or translocations, and by next generation sequencing based off-target cleavage assays and found to lack evidence of unintended outcomes.

As shown in FIGS. 2A-2C, constructs containing genes of interest were inserted into endogenous loci. This was accomplished with the use of homologous repair templates containing the coding sequence of the gene of interest flanked by left and right HR arms. In addition to the HR arms, the gene of interest was sandwiched between 2A peptides, a protease cleavage site that is upstream of the 2A peptide to remove the 2A peptide from the upstream translated gene of interest, and signal sequences (FIG. 2B). Once integrated into the genome, the gene of interested expression gene cassette was transcribed as single messenger RNA. During the translation of this gene of interest in messenger RNA, the flanking regions were unlinked from the gene of interest by the self-cleaving 2A peptide and the protease cleavage site was cleaved for the removal of the 2A peptide upstream from the translated gene of interest (FIG. 2C). In addition to the 2A peptide and protease cleavage site, a gly-ser-gly (GSG) linker was inserted before each 2A peptide to further enhance the separation of the gene of interest from the other elements in the expression cassette.

It was determined that P2A peptides were superior to other 2A peptides for Cell Products because of its efficient cleavage. Accordingly, two (2) P2A peptides and codon divergence were used to express the gene of interest without introducing any exogenous epitopes from remaining amino acids on either end of the gene of interest from the P2A peptide. The benefit of the gene edited cell having no exogenous epitopes (i.e., no flanking P2A peptide amino acids on either side of the gene of interest) is that immunogenicity is drastically decreased and there is less likelihood of a patient infused with a Cell Product containing the gene edited cell to have an immune reaction against the gene edited cell.

As described in PCT/US/2018/058230, NeoTCRs were integrated into the TCRα locus of T cells. Specifically, a homologous repair template containing a NeoTCR coding sequence flanked by left and right HR Arms was used. In addition, the endogenous TCRβ locus was disrupted leading to the expression of only TCR sequences encoded by the NeoTCR construct. The general strategy was applied using circular HR templates as well as with linear templates.

The neoantigen-specific TCR construct design is diagrammed in FIGS. 3A and 3B. The target TCRα locus (Ca) is shown along with the plasmid HR template, and the resulting edited sequence and downstream mRNA/protein products are shown. The target TCRα locus (endogenous TRAC) and its CRISPR Cas9 target site (horizontal stripe, cleavage site designated by arrow) are shown (FIG. 3A). The circular plasmid HR template with the polynucleotide encoding the NeoTCR, which is located between left and right homology arms (“LHA” and “RHA” respectively), is shown (FIG. 3A). The region of the TRAC introduced by the HR template that was codon optimized is shown (vertical stripe). The TCRβ constant domain was derived from TRBC2, which is indicated as being functionally equivalent to TRBC1. Other elements in the NeoTCR cassette include: 2A=2A ribosome skipping element (by way of non-limiting example, the 2A peptides used in the cassette are both P2A sequences that are used in combination with codon divergence to eliminate any otherwise occurring non-endogenous epitopes in the translated product); P=protease cleavage site upstream of 2A that removes the 2A tag from the upstream TCRβ protein (by way of non-limiting example the protease cleavage site can be a furin protease cleavage site); SS =signal sequences (by way of non-limited example the protease cleavage site can be a human growth hormone signal sequence). The HR template of the NeoTCR expression gene cassette includes two flanking homology arms to direct insertion into the TCRα genomic locus targeted by the CRISPR Cas9 nuclease RNP with the TCRα guide RNA. These homology arms (LHA and RHA) flank the neoE-specific TCR sequences of the NeoTCR expression gene cassette. While the protease cleavage site used in this example was a furin protease cleavage site, any appropriate protease cleavage site known to one of skill in the art could be used. Similarly, while HGH was the signal sequence chosen for this example, any signal sequence known to one of skill in the art could be selected based on the desired trafficking and used.

Once integrated into the genome (FIG. 2C), the NeoTCR expression gene cassette is transcribed as a single messenger RNA from the endogenous TCRα promoter, which still includes a portion of the endogenous TCRα polypeptide from that individual T cell (FIG. 2C). During ribosomal polypeptide translation of this single NeoTCR messenger RNA, the NeoTCR sequences are unlinked from the endogenous, CRISPR-disrupted TCRα polypeptide by self-cleavage at a P2A peptide (FIG. 2C). The encoded NeoTCRα and NeoTCRβ polypeptides are also unlinked from each other through cleavage by the endogenous cellular human furin protease and a second self-cleaving P2A sequence motifs included in the NeoTCR expression gene cassette (FIG. 2C). The NeoTCRa and NeoTCRβ polypeptides are separately targeted by signal leader sequences (derived from the human growth hormone, HGH) to the endoplasmic reticulum for multimer assembly and trafficking of the NeoTCR protein complexes to the T cell surface. The inclusion of the furin protease cleavage site facilitates the removal of the 2A sequence from the upstream TCRβ chain to reduce potential interference with TCRβ function. Inclusion of a gly-ser-gly linker before each 2A (not shown) further enhances the separation of the three polypeptides.

Additionally, three repeated protein sequences are codon diverged within the HR template to promote genomic stability. The two P2A are codon diverged relative to each other, as well as the two HGH signal sequences relative to each other, within the TCR gene cassette to promote stability of the introduced NeoTCR cassette sequences within the genome of the ex vivo engineered T cells. Similarly, the re-introduced 5′ end of TRAC exon 1 (vertical stripe) reduces the likelihood of the entire cassette being lost over time through the removal of intervening sequence of two direct repeats.

In addition to NeoTCR Products, this method can be used for any Cell Product.

FIG. 3 shows the results of an In-Out PCR confirming precise target integration of the NeoE TCR cassette. Agarose gels show the results of a PCR using primers specific to the integration cassette and site generate products of the expected size only for cells treated with both nuclease and DNA template (KOKI and KOKIKO), demonstrating site-specific and precise integration.

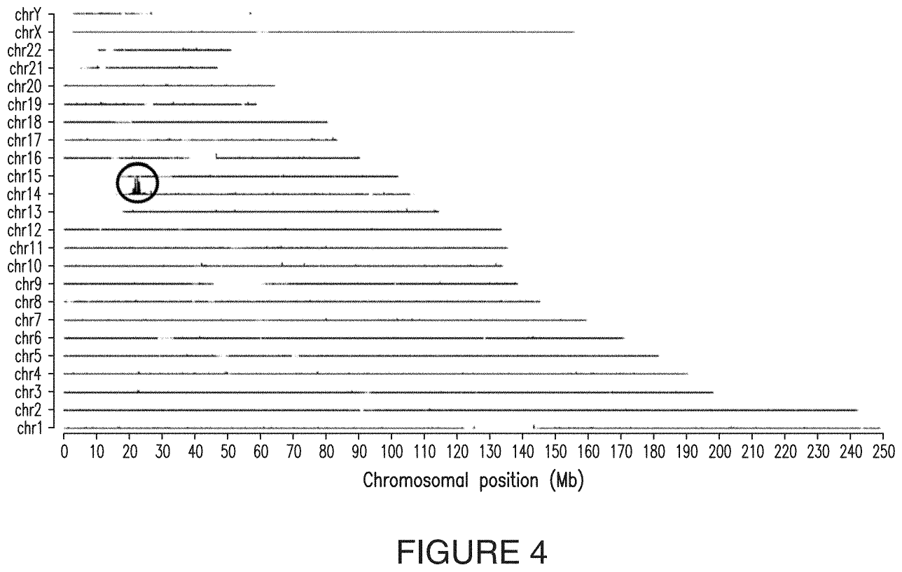

Furthermore, as shown in FIG. 4, Targeted Locus Amplification (TLA) was used to confirm the specificity of targeted integration. Crosslinking, ligation, and use of primers specific to the NeoTCR insert were used to obtain sequences around the site(s) of integration. The reads mapped to the genome are binned in 10 kb intervals. Significant read depths were obtained only around the intended site the integration site on chromosome 14, showing no evidence of common off-target insertion sites.

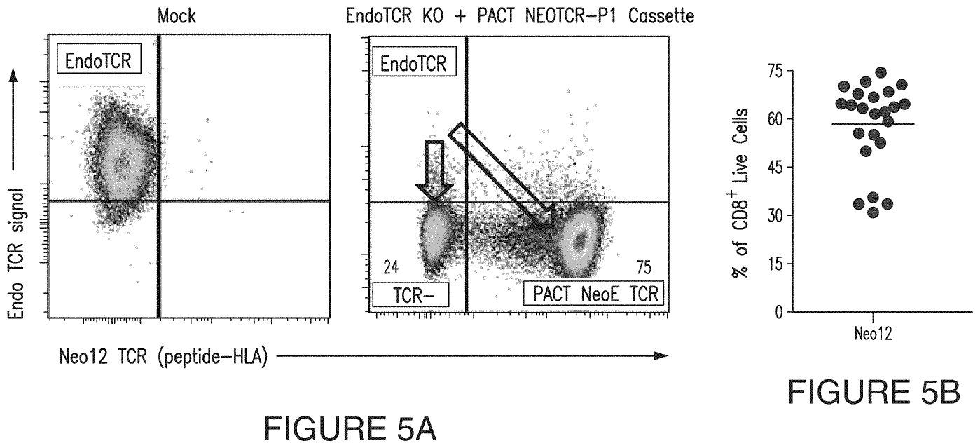

Antibody staining for endogenous TCR and peptide-HLA staining for neoTCR reveals that the engineering results in high frequency knock-in of the NeoTCR, with some TCR- cells and few WT T cells remaining (FIG. 5A). Knock-in is evidenced by neoTCR expression in the absence of an exogenous promoter. Engineering was carried out multiple times using the same neoTCR with similar results (FIG. 5B). Therefore, efficient and consistent expression of the NeoTCR and knockout of the endogenous TCR in engineered T cells was achieved.

### Example 2

### Expression and Phenotype Characterization of Engineered T Cells (i.e., NeoTCR Cells)

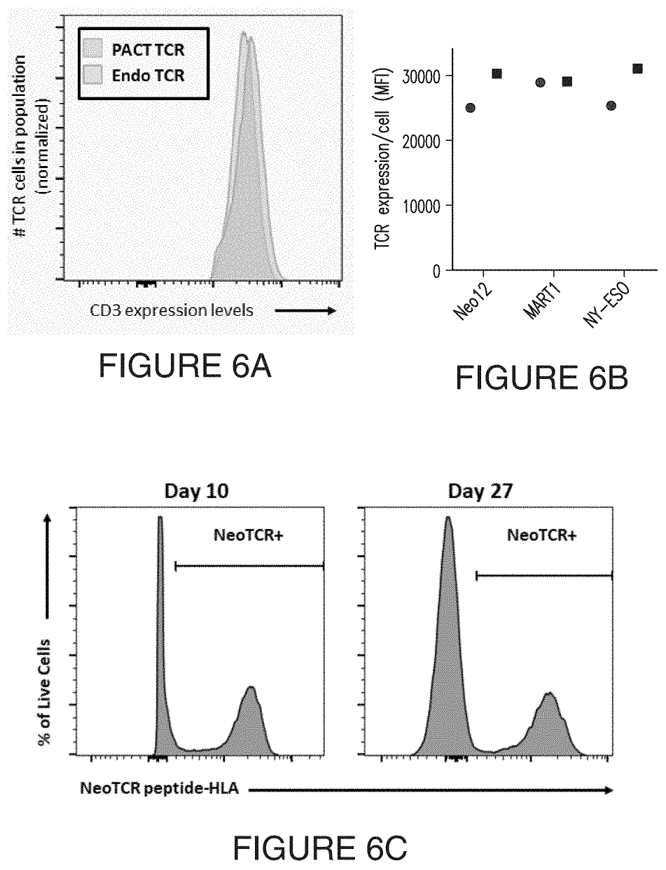

Engineered NeoTCRs (TCRs that have been introduced into the endogenous TRAC locus) were expressed at endogenous levels reproducibly for multiple different TCRs and expression was maintained without detriment to the cells. Lack of competing with the endogenously expressed TCR removed competition for CD3 subunits, allowing expression of the neoTCR at levels similar to the native TCRs (FIG. 6A). Accordingly, as shown in FIG. 2A, the endogenous TCRs were knocked out to allow for the sole expression of the NeoTCR. Consistent levels of NeoTCR expression was observed regardless of the TCR identity (FIG. 6B). Cells assayed for NeoTCR expression by dextramer staining showed similar rates of editing and NeoTCR expression levels were maintained at 10 and 27 days post engineering, suggesting no competitive disadvantage to the engineered T cells and no silencing of the expression cassette (i.e., the NeoTCR inserted into the TRAC locus of the T cell as described in Example 1) (FIG. 6C).

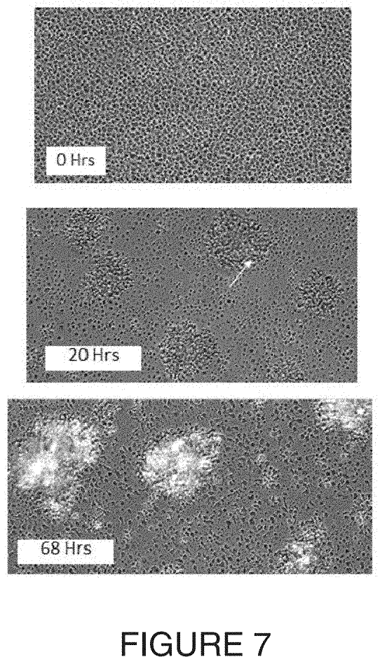

Expression from the integrated cassette (i.e., the NeoTCR in NeoTCR Products) occured within 3 days. To visualize the expression patterns and time frames of the NeoTCRs post gene editing, the cells were modified (i.e., gene edited) to express mCherry in tandem with the neoTCR and monitored using time-lapse fluorescence microscopy. Cells showed high levels of mCherry expression 2-3 days post-modification (FIG. 7). Thus, the flexibility and the ability to efficiently engineer cells with additional components were also demonstrated. Accordingly, additional cargo (e.g., one or more additional proteins) can be encoded in the cassette and incorporated into the cells using the gene editing methods described herein resulting in a cell that expresses a NeoTCR and one or more additional genetically encoded elements such a protein.

### Example 3

### Phenotype and Manufacturing of Engineered T Cells (i.e., NeoTCR Cells)

Phenotype of engineered T cells (i.e., NeoTCR cells). Engineered NeoTCR T cells (e.g., NeoTCR Products) are highly functional as demonstrated by antigen-specific proliferation, killing and cytokine production. Phenotype and detailed functional characterization of the NeoTCR Products made using the methods described herein were performed as described below.

T cell subset distribution was analyzed by standard flow cytometry. Surface profiling of CD8 T cells upon contact with cognate or irrelevant target cells was performed. Briefly, T cells were co-cultured and harvested at indicated time points (4 h, 24 h, 48 h, and 72 h), washed in PBS, stained with a viability dye, an anti-CD8 antibody and a T cell activation panel composed of 83 markers.

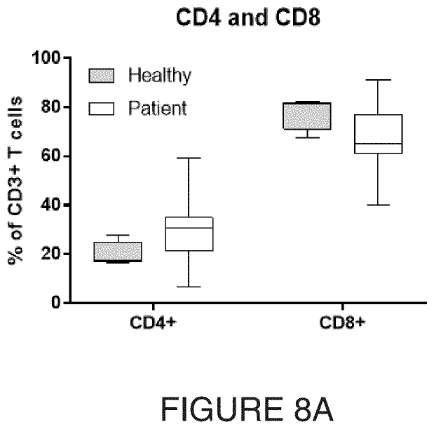

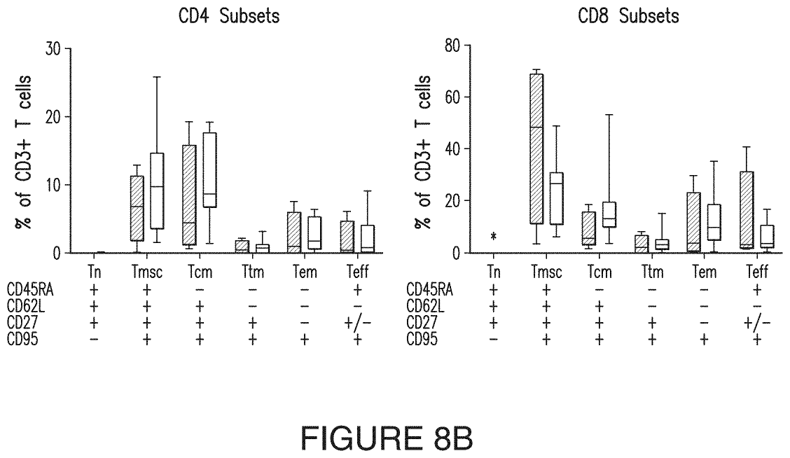

Total CD4 and CD8 subset distribution was determined after lab-scale manufacturing from blood of healthy donors (black) or patients with cancer (gray) day 13 (FIG. 8A). CD4 T cell and CD8 T cell phenotype after expansion were predominantly T memory stem cells (Tmsc) and central memory T cells (Tcm) (FIG. 8B). Specifically, FIG. 8B shows CD4 (left panel) and CD8 subset distribution after laboratory-scale manufacturing from blood of healthy donors (black) or patients with cancer (gray). CD4 T cell and CD8 T cell subset phenotypes in the final product are predominantly Tmsc and Tcm (both populations are CD62L^{high}). In subsets are not visible on the graph as percentages are <1% after activation of T cells during manufacturing process. * p<0.05. Healthy donors: n=4 from 3 unique donors; patients: n=14 from 8 unique donors. Gating strategy: single cells, live cells, CD3+ cells, phenotype subsets. Markers used for subset distribution definition and analysis are indicated below the graphs.

Therefore, it was demonstrated that NeoTCR Products are mainly of the “Younger” Tscm and Tcm phenotype.

According to the linear model of T cell differentiation, naïve cells differentiate into memory stem cell (T_{MSC}) and central memory (T_{CM}) cell phenotypes upon initial activation with appropriate signals from antigen-presenting cells. These ‘younger’ or less-differentiated T cell populations have been shown to engraft well into lymphocyte-depleted animals, plus to proliferate vigorously on further stimulation while maintaining the memory cell pool for persistence (Klebanoff, Gattinoni and Restifo, 2012). The model for linear differentiation of T cell subsets is presented in Table 2.

TABLE 2
Model for linear differentiation of T cell subsets

| T cell subset | Cytokine profile | Functional characteristics |
|---|---|---|
| Naive (T_{N}) | CD45RA+ CD62L+, | Weak effector T cell function |
| | CD28+ CD95− | Robust proliferation |
| | CCR7+ CD27+ | Robust engraftment |
| Memory stem | CD45RA + CD62L+, | Long telomeres |
| cell (T_{MSC}) | CD28+ CD95+ | |
| | CCR7+ CD27+ | |
| Central memory | CD45RO+ CD62L+, | |
| (T_{CM}) | CD28+ CD95+ | |
| | CCR7+ CD27+ CD127+ | |
| Effector memory | CD45RO+ CD62L−, | Rapidly execute effector |
| (T_{EM}) | CD28+/−CD95+ | functions e.g., IFN-γ, TNF-α, |
| | CCR7− CD27− | lysis of target cells |
| | Busch: CD27+ CD127+ | Limited proliferation |
| Effector (T_{E}) | CD45RO+ CD62L−, | Low engraftment potential |
| | CD28+/−CD95+ | Short telomere length |
| | Perforin^{hi}Gzmb^{hi} | |
| | CCR7−CD27− | |

These ‘younger’ populations, however, do not secrete the full cytokine and effector protein cascade observed from more differentiated or ‘older’ effector memory (TEM) and effector T cells (T_{E}) upon encountering cognate neoE-HLA expressing targets. However, while the ‘older’ cells are highly effective for killing target tumor cells, they lack the capacity to proliferate and to persist. In this manner, they have become terminally differentiated for the purpose of killing target T cells upon activation.

Published reports from animal models and clinical studies have suggested that adoptive cell therapies comprising T cells with ‘younger’ or less-differentiated memory stem cell phenotypes achieve an improved overall response and clinical outcome than studies where ‘older’ or more-differentiated T effector cells were administered. Data from mouse models and in clinical trials of CD19-targeted CAR-T cells demonstrated a correlation of the younger phenotype with cell persistence and overall response rate (Busch et al, 2018); (Sabatino et al., 2016). Thus, the administration of ‘younger’ T cell phenotypes is of potentially higher benefit to patients with cancer, and this is associated with improved engraftment potential, prolonged persistence post infusion, and rapid differentiation into effector T cells upon exposure to their cognate antigen. These properties highlight the value of infusing neoTCR-expressing T cells comprising T memory stem cell (T_{MSC}) and T central memory (T_{CM}) phenotypes.

The manufacturing process described herein was deliberately developed to favor the generation of T cell populations of the less-differentiated phenotype. The composition of T cell phenotypes resulting from the NeoTCR Product cell manufacturing process was interrogated by flow cytometric analysis. As desired, NeoTCR cells of memory stem cell and central memory phenotypes represent significant T cell phenotypes in the NeoTCR Product profile.

Together, these ex vivo mechanism of action studies described herein demonstrated that the NeoTCR cells generated from healthy donors or patients with cancer that were formulated into NeoTCR Products consisted significantly of CD8+ and CD4+ T cells of the desired younger phenotype subsets (T_{MSC} and T_{CM}). Upon encounter of cognate peptide-HLA, these cells rapidly transition into polyfunctional effector cells that demonstrated potent cytokine production, tumor killing activity and proliferative capacity, with the potential to eradicate tumor cells throughout the body. Manufacture of engineered T cells (i.e., NeoTCR cells). In order to manufacture a product that consists significantly of CD8+ and CD4+ T cells of the desired younger phenotype subsets (T_{MSC} and T_{CM}), specific manufacturing methods had to be tested to find the correct conditions for such a phenotype. This was not an obvious endeavor considering that cell therapies currently in clinical trials and available for commercial sale are predominantly made up of effector cells that will not engraft in a patient, will die, and will require repeated infusion for continued tumor killing.

An exemplary first step of the manufacturing process is to collect a leukopak from the cancer patient for whom a NeoTCR Product is designed, manufactured and administered to the patient for the treatment of cancer. The patient's cells, e.g., from a leukopak, are then processed to enrich for CD4 and CD8 T cells. In certain instances, trace amounts of other cell types and impurities persist in the CD4 and CD8 T cell fraction; however, the CD4 and CD8 T cells within that fraction are the predominant cell type.

The second step of the manufacturing process is to activate the CD4 and CD8 T cells. This enrichment can be achieved, e.g., using a media such as TransACT (Miltenyi), with any other non-bead-based reagent, or with a bead-based reagent. Depending on the method of activating the CD4 and CD8 T cells, the cells are cultured for an appropriate time to allow for optimal activation. For example, when the cells are activated with TransACT, they were cultured for 48 hours (i.e., 2 days).

The third step of the manufacturing process is to engineer (i.e., the gene editing methods described in Example 1) the CD4 and CD8 T cells to express a NeoTCR (and other cargo if desirable) following the activation. As described above, when the cells were activated with TransACT, the engineering occurred on day 2.

The fourth step of the manufacturing process is to culture the engineered NeoTCR cells to allow the cells to expand in media and take on young phenotype. The T cell growth media used for this step was supplemented with 3% human AB serum, 12.5 ng/mL IL7, and 12.5 ng/mL IL15 for the remainder of the manufacturing period. One such media that can be used for this manufacturing process is the TexMACS® GMP media.

The fifth and final step of the manufacturing process is to package the NeoTCR cells into a cryobag (or other suitable container) for storage followed by administration to the cancer patient. In certain embodiments, the cells are harvested from the previously described culture conditions by washing the cells in Plasma-Lyte A supplemented with 2% HAS (w/v) and then concentrated and eluted. Following the harvest, the cells are then formulated for storage in 46% Plasma-Lyte A, 1% HSA (w/v), and 50% CryoStor CS10 in CryoMACS bags for cryopreservation and later thawed and administered to the patient for whom the NeoTCR Product was made.

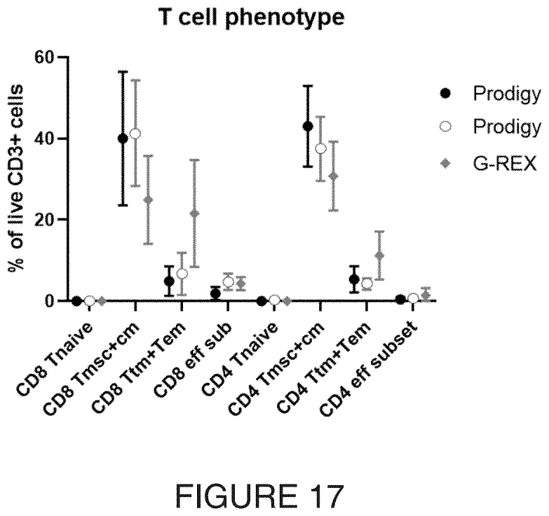

Experiments were then performed to determine the phenotype of the cells. As shown in FIG. 17, the majority of the CD8 T cells and CD4 T cells were found to be either Tmsc or Tcm cells (i.e., having a Tmsc or Tcm phenotype).

Selection of cytokine concentrations. In order to design a cell culture media that was conducive to growing T cells to promote the Tmsc and Tcm state, experiments were conducted to determine the proper amounts of cytokines for the cell media. The cytokine conditions tested are provided in Table 3.

TABLE 3
Groups for activation pre-transfection

| Group | Cells | Volume | Conditions | Transfection Conditions |
|---|---|---|---|---|
| 1 | 5 E6 | 100 μL | No cytokines | Neo12 + RNPα + RNPβ |
| 2 | 5 E6 | 100 μL | IL-2 5 ng | Neo12 + RNPα + RNPβ |
| 3 | 5 E6 | 100 μL | IL-2 50 ng | Neo12 + RNPα + RNPβ |
| 4 | 5 E6 | 100 μL | IL-2 500 ng | Neo12 + RNPα + RNPβ |
| 5 | 5 E6 | 100 μL | IL-15 12.5 ng | Neo12 + RNPα + RNPβ |
| 6 | 5 E6 | 100 μL | IL-2 (20 ng), IL7 | Neo12 + RNPα + RNPβ |
| | | | (12.5 ng), IL-15 (12.5 ng) | |
| 7 | 5 E6 | 100 μL | IL-2 (20 ng), IL7 | Neo12 + RNPα + RNPβ |
| | | | (12.5 ng) | |
| 8 | 5E6 | 100 μL | IL2 (20 ng), IL-15 | Neo12 + RNPα + RNPβ |
| | | | (12.5 ng) | |
| 9 | 5E6 | 100 μL | IL7 (12.5 ng), IL-15 | Neo12 + RNPα + RNPβ |
| | | | (12.5 ng) | |

Based on cell expansion and viability assays, transgene expression assays, and T cell phenotype (including cell exhaustion markers such as LAG-3 and 4-1BB) assays, each using the conditions described in Table 3, it was determined that the optimal amount of IL-2 was 20 ng, the optimal amount for IL-7 was 12.5 ng, and the optimal amount of IL-15 was 12.5ng. Additional concentrations of IL-7 and IL-15 were tested (data not shown).

Cell culture and manufacture conditions for young T cells. In order to allow NeoTCR Products to engraft culture media the promoted the cells to stay in a Tmsc and Tcm state was needed. Further, there was interest in finding a culture condition that would not make the T cells dependent on IL-2 when administered to patients (i.e., allow for the administration of the NeoTCR Product without necessarily requiring the co-administration of IL-2). Experiments were designed to assess the functional impact of culturing gene edited T cells with 1) IL2 during activation and expansion, 2) IL-7+IL-15 during activation and expansion or 3) IL2 during activation and IL-7+IL-15 during expansion. The experiments consisted of the following steps over the course of 14 days: a) Day 0: Thaw previously frozen patient derived T cells, b) Day 1: perform CD3 negative selection and activation, c) Day 3: transfect the cells with a NeoTCR and plate on a 24-well G-Rex plate, d) Day 3-14: cytokine treatment and expansion using conditions 1-3 described above, e) Day 14: divide the cells in three parts for part 1 to be stained with dextramer to determine the transfection efficiency, part 2 for freezing and storage, and part 3 for functional assays. The functional assays performed included cell count/viability, proliferation, cell killing, cytokine production, Incycte assay, and transgene expression. The NeoTCRs used for transfection were neo12, F5, and 1G4. The cytokine treatments performed are described in Table 4 below.

TABLE 4
Cytokine treatments

| Experiment | Cytokine Concentration | Transfection Conditions |
|---|---|---|
| 1 | 12.5 ng/mL IL-7, | TCRα & TCRβ RNP + HR DNA |
| | 12.5 ng/mL IL-15 | (1G4 TCR) |
| 2 | 12.5 ng/mL IL-7, | TCRα & TCRβ RNP + HR DNA |
| | 12.5 ng/mL IL-15 | (neop12 TCR) |
| 3 | 12.5 ng/mL IL-7, | TTCRα & TCRβ RNP + HR DNA |
| | 12.5 ng/mL IL-15 | (F5 TCR) |
| 4 | 12.5 ng/mL IL-7, | Mock (no transfection) |
| | 12.5 ng/mL IL-15 | |
| 5 | 11,2 pre-EP then | TCRα & TCRβ RNP + HR DNA |
| | 12.5 ng/mL IL-7, | (1G4) |
| | 12.5 ng/mL IL-15 | |
| 6 | IL-2 pre-EP then | TCRα & TCRβ RNP + HR DNA |
| | 12.5 ng/mL IL-7, | (neop12 TCR,) |
| | 12.5 ng/mL IL-15 | |
| 7 | IL-2 pre-EP then | TCRα & TCRβ RNP + HR DNA |
| | 12.5 ng/mL IL-7, | (F5 TCR) |
| | 12.5 ng/mL IL-15 | |
| 8 | IL-2 pre-EP then | Mock (no transfection) |
| | 12.5 ng/mL IL-7, | |
| | 12.5 ng/mL IL-15 | |
| 9 | 20 ng IL-2 | TCRα & TCRβ RNP + HR DNA |
| | | (1G4 TCR) |
| 10 | 20 ng IL-2 | TCRα & TCRβ RNP + HR DNA |
| | | (neop12 TCR) |
| 11 | 20 ng IL-2 | TCRα & TCRβ RNP + HR DNA |
| | | (F5 TCR) |
| 12 | 20 ng IL-2 | Mock (no transfection) |
| 13 | 20 ng IL-2 | TCRα RNP only control (no TCR) |
| 14 | 20 ng IL-2 | TCRβ RNP +HR DNA (F5 TCR) |
| | | control (no TCRα) |

These experiments showed similar gene editing efficiency among the different TCR (neo12, F5 and 1G4) as well as among the different cytokine conditions used during activation and expansion. Similar cell counts were observed on day 14 independently on the cytokines used during activation and expansion.

Antigen-specific killing was tested at 24 and 48hours while antigen-specific T cells proliferation was assessed at 72 hours. As control, mock T cells (wild type TCR) were tested against K562 cells pulsed with MART12 peptide or against K562 constitutively expressing MART1-HLA-A02 complex. Edited T cells were tested against K562 tumor cells expressing HLA-A02 and then pulsed with different amount of cognate peptide for 1 hour or against K562 constitutively expressing the specific peptide-HLA-A02 complex. Antigen-specific cytokine secretion was assessed in the supernatant of the killing assay using the Cytometric Bead Assay (CBA) (Table 5). Edited F5 and neo12 TCR T cells demonstrated antigen-specific target cell killing, proliferation and cytokine secretion. Edited 1G4 TCR T cells demonstrated antigen-specific target proliferation and cytokine secretion but showed K562 target cell killing regardless of whether K562 cells were pulsed with cognate peptide or constitutively expressed NYESO-HLA-A02 complex.

TABLE 5
Summary of the different conditions tested in the CBA assay

| Edited T cells | Cytokines | Time point for CBA |
|---|---|---|
| F5 T cells | IL7 + 1L15 | 24 |
| neo12 T cells | IL7 + IL15 | 24 |
| F5 T cells | IL7 + IL15 | 48 |
| 1G4 T cells | IL7 + IL15 | 48 |
| neo12 T cells | IL7 + 1L15 | 48 |
| F5 T cells | IL-2 and expansion in IL7 + IL15 | 48 |
| 1G4 T cells | IL-2 and expansion in IL7 + IL15 | 48 |
| neo12 T cells | IL-2 and expansion in IL7 + IL15 | 48 |
| F5 T cells | IL-2 | 48 |
| 1G4 T cells | IL-2 | 48 |
| neo12 T cells | IL-2 | 48 |

The Incucyte co-culture killing assay with was set up on day 14 using a 4:1 P:T (P=Product, total T cells; T=target cells) ratio of neo12 TCR T cells (not labeled cells) incubated with K562 constitutively expressing neo12-HLA-A02 complex (green as these cells also express Zgreen). To detect cell apoptosis in real time, a highly-selective phosphatidylserine (PS) cyanine fluorescent dyes (IncuCyte Annexin V) was added to the coculture. Addition of this reagent to normal healthy cells does not perturb cell growth or morphology. Once cells become apoptotic, plasma membrane PS asymmetry is lost. PS exposure on the extracellular surface enables binding of Annexin V resulting in a bright and photostable fluorescent signal. Images were collected at 4 hours interval for several days using a 10× objective. Antigen-specific cytotoxic activity and proliferation by neo12-TCR T cells was observed when neo12 T cells were co-cultured with K562 target cells expressing cognate neo12 peptide-HLA but not when co-cultured with K562 target cells expressing an irrelevant peptide.

Antigen-specific proliferation of neo12 T cells (not labeled in this assay) was demonstrated by the increased numbers of unlabeled cells over the course of the assay.

This experiment demonstrated that the use of different cytokine combinations (IL-2, IL-2 during pre-electroporation then IL-7 plus IL-15 or always IL-7 plus IL-15) did not have a major effect on the cell growth, gene editing, or functionality of the edited T cells.

### Example 4

### Functional Characterization of Engineered T Cells

Successfully engineered NeoTCR cells were shown to traffic to tissues harboring tumor cells presenting the neoantigen peptide in the context of the autologous cognate HLA receptor. Recognition of the cognate neoE-HLA complexes triggered T cell proliferation and secretion of effector molecules from the engineered T cells.

To demonstrate these activities in the engineered cells T cells provided herein, ex vivo mechanism-of-action studies were performed by generating NeoTCR cells derived from the blood of healthy donors (i.e., patients without cancer) or patients with cancer. T cells were engineered to express two model TCRs: neo12, a neoTCR isolated from a melanoma patient's PBMCs using the imPACT Isolation Technology described in PCT/US2020/17887 (which is herein incorporated by reference in its entirety), and F5 TCR, a clinically validated TCR against the tumor antigen MART1. Phenotypic analysis was performed to characterize the T cell subset distribution of the NeoTCR-P1 final cell product. Antigen-specific activity was characterized by measuring target-specific killing, proliferation and cytokine production.

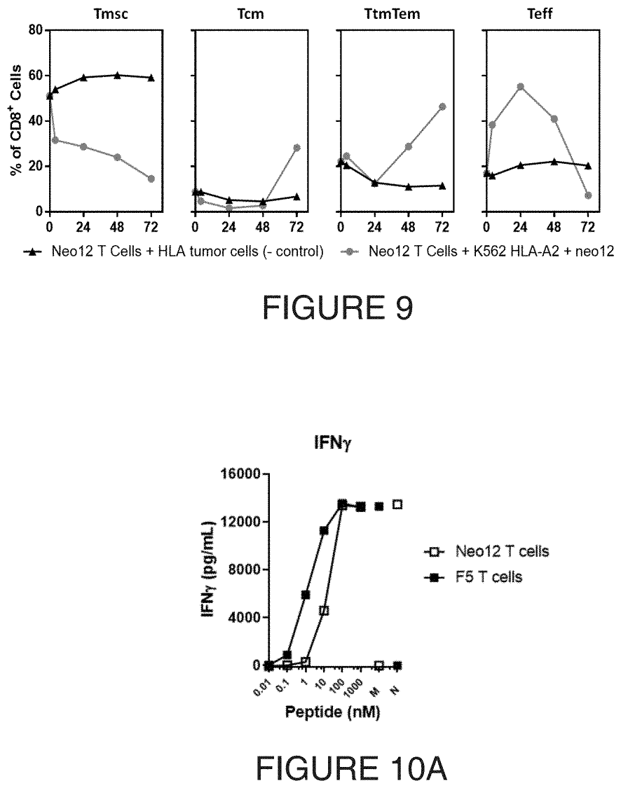

NeoTCR T cells rapidly convert to effector cells on antigen exposure. NeoTCR cells (from a NeoTCR Product) expressing the neo12 TCR were co-cultured with tumor cells pulsed with cognate peptide (K562 neo12 peptide-HLA-A2 displaying tumor cells, red circle) for up to 72 hours. As shown in FIG. 9, upon cognate antigen encounter, NeoTCR cells rapidly differentiate into potent effector T cells. No changes were observed when NeoTCR cells were co-cultured with tumor cells alone (K562 HLA-A2 displaying, negative control tumor cells, black triangles). The 0-hour time point was T cells alone.

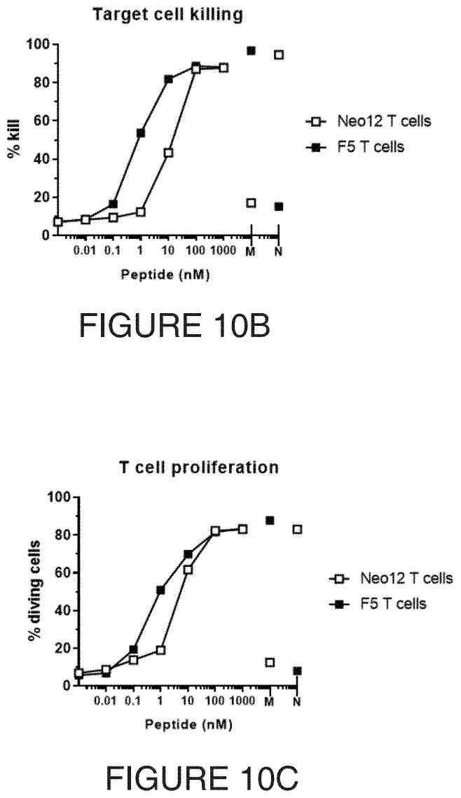

Antigen-Specific Activity of Precision Genome Engineered Human NeoTCR cells. CD8 and CD4 T cells from healthy donors or patients with cancer were precision genome engineered (as discussed in Example 1) to express the neo12 TCR or the F5 TCR. NeoTCR T cells expressing Neo12 TCR or F5 (MART1 TCR) showed functional activity as measured by antigen-specific IFNγ cytokine secretion (FIG. 10A), target cell killing (FIG. 10B) and, proliferation (FIG. 10C).

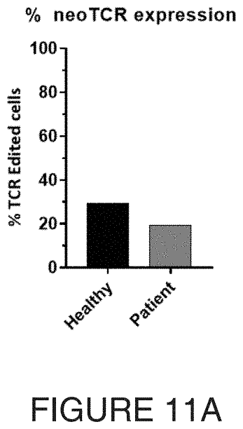

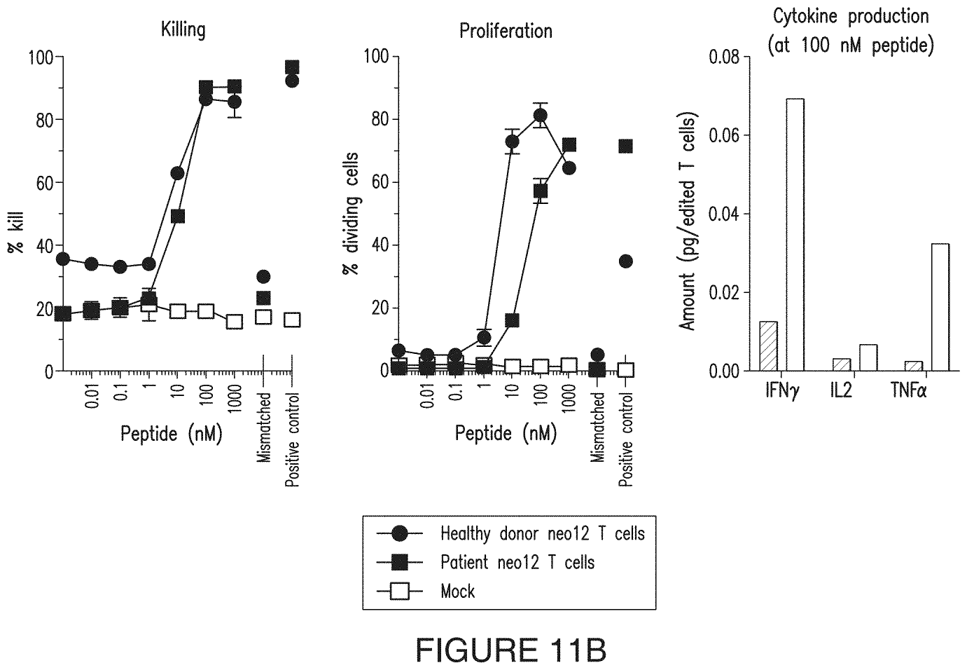

Comparable antigen-specific activity of NeoTCR cells derived from patients with cancer or healthy donors. NeoTCR-cells expressing neo12 TCR generated from a patient with cancer (melanoma) or a healthy donor showed comparable gene-editing efficiency (% of neoTCR expression; FIG. 11A), and functional activity as measured by target cell killing, proliferation and cytokine production (IFNγ, IL2 and TNFα) measured in the supernatant using the cytokine bead assay (FIG. 11B). Mismatched experiments were also performed to demonstrate the specificity of the NeoTCR cells. Specifically, surrogate tumor target cells expressing MART1-HLA-A2 complex were paired with the neo12 TCR cells. There was no cell killing, proliferation, or cytokine production for the mismatched tumor-cell pairing (i.e., there waso activity was observed with mock control T cells). Accordingly, only the properly matched tumor responded to exposure to the NeoTCR cells (i.e., surrogate tumor cells expressing neo12-HLA-A2 complex responded as shown by cell killing, proliferation of the NeoTCR cells, and cytokine production when exposed to the properly matched neo12 (HLA-A2 complex) NeoTCR cells.

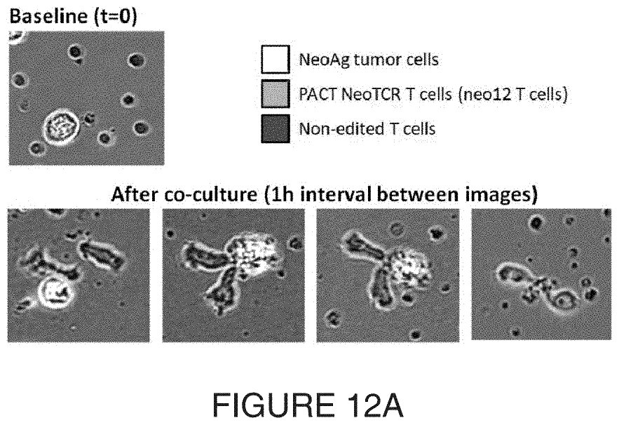

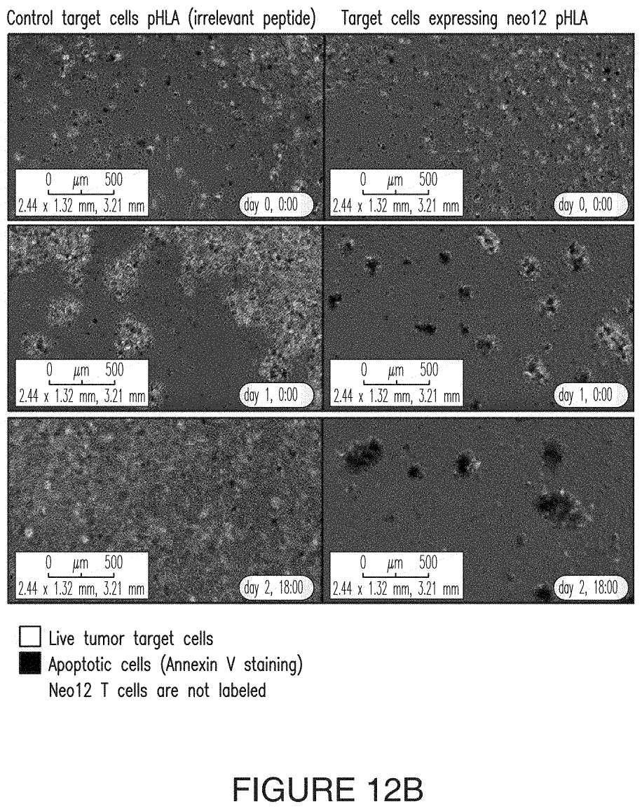

Specific killing of antigen-expressing surrogate tumor target cells and antigen-specific proliferation of NeoTCR T cells. NeoTCR cells that were designed and engeineered (i.e., gene edited) to express mCherry (red) and the neo12 TCR were co-cultured with tumor cells expressing ZsGreen and the specific neoantigen (neo12) and HLA-A02 complex (i.e., the cognate antigen to the neo12 TCR) (FIG. 12A). At baseline, edited (red) and non-edited T cells (grey) were round and smaller in size than tumor cells (green). After encountering antigen-expressing tumor cells, the neoTCR T cells (expressing the neo12 TCR and mCherry) became elongated, formed immunological synapses and killed the target tumor cell. The non-edited T cells did not show any cytotoxic activity. The images shown in FIG. 12A were taken at lh intervals.

Timelapse microscopy of tumor cell death and T cell proliferation. NeoTCR cells that were designed and engineered (i.e., gene edited) to express the neo12 neoTCR (also referred to as Neo12 TCR-T cells herein) were co-cultured with K562 tumor cells transfected to express irrelevant (i.e., antigen-HLA complex that is not cognate to the neo12 neoTCR) peptide-HLA-A2 protein complexes on the surface (left column) or K562-neo12-HLA-A2 expressing cells (i.e., the cognate antigen-HLA complex) (right panel) (FIG. 12B). Tumor cells also expressed a variant of green fluorescent protein (GFP or ZsGreen) stably and homogeneously. Images were collected over 48 h and shown in FIG. 12B at time 0 (top panels), 24 h (middle panels) and 48 h (bottom panels). To detect real time apoptosis, a highly-selective phosphatidylserine cyanine fluorescent dye (IncuCyte Annexin V in red) was added to the co-culture. While T cells were not labeled in the experiment described herein and shown in FIG. 12B, antigen-specific proliferation is demonstrated and appreciated visually by the increased numbers of T cells over 2 days (right column).

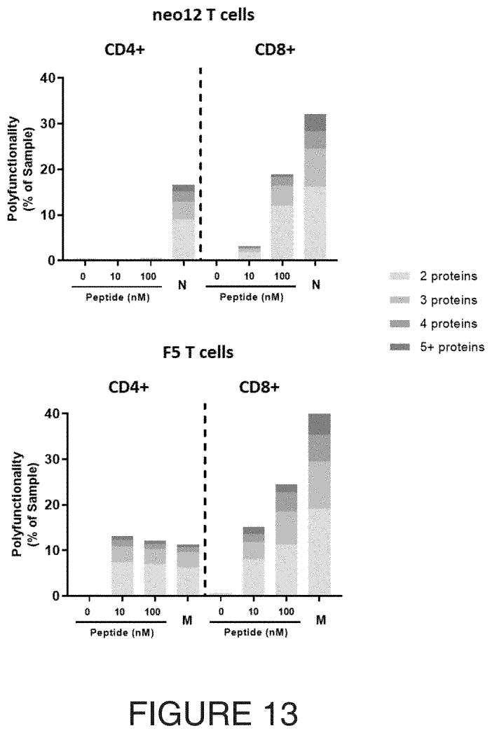

CD4 and CD8 NeoTCR-P1 T cells are polyfunctional. Graphs showing the percentage of CD4 and CD8 NeoTCR cells that were engineered to either express the neo12 TCR or F5 TCR and secreting 2, 3, 4 or greater than- equal to 5 cytokines uponencountering cognate antigen are shown in FIG. 13. These NeoTCR cells were co-cultured with target cells pulsed with no peptide, 10 nM or 100 nM specific peptide (i.e., the cognate antigen-HLA complex to either neo12 NeoTCR cells or the F5 NeoTCR cells), or with target cells constitutively expressing peptide-HLA on their surface (as shown in FIG. 13, N=neo12 HLA-A2 cells; M=MART1 HLA-A2 cells).

Specifically, NeoTCR cells were co-cultured with K562 cells expressing HLA-A02 pulsed with different concentrations of peptides (neo12 or F5, 0-1000 nM) or with K562 cells constitutively expressing peptide-HLA complex at a final Product to Target ratio (P:T) ratio of 4:1. Cytokine secretion was measured in the cell supernatant at 24 h using the BD Cytokine Bead Array (CBA) Human Th1/Th2 Cytokine Kit II. Target cell killing and T cell proliferation were evaluated at 48 h and 72 h, respectively.

Secreted cytokine levels were assessed after 24 hours of co-culture using IsoPlexis single-cell secretome analysis. Specifically, NeoTCR cells were cultured for 24 h and then loaded onto a single-cell barcode chip containing 12000 microchambers pre-patterned with a 32-plex antibody array. The NeoTCR cells were imaged to identify single-cell locations and incubated for an additional 16 h. Single-cell cytokine signals were then captured and digitized with a microarray scanner. The polyfunctionality (2+ cytokines per cell) and polyfunctional strength index (PSI) of single CD4+ and CD8+ T cells was evaluated. Cells secreting 2 or more cytokines were considered polyfunctional.

The efficiency of gene editing of the neo12 TCR and the F5 neoTCR into the total T cell population was ˜40%.

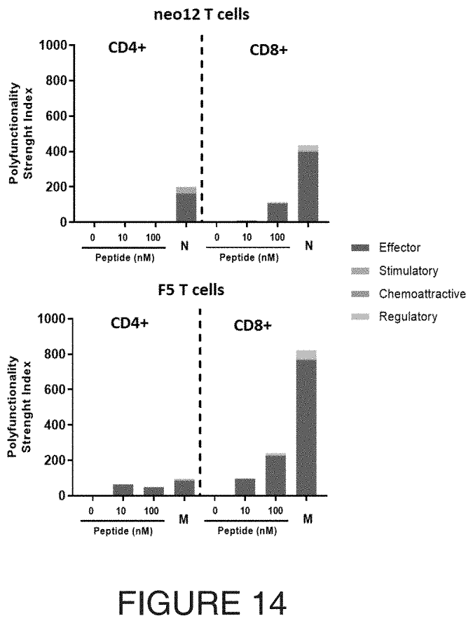

NeoTCR P-1 polyfunctional responses are strongly driven by proteins associated with effector function. Polyfunctional strength index (PSI) is defined as the number of T cells secreting greater than 2 effector molecules per cell (polyfunctional T cells in FIG. 13), multiplied by mean fluorescence intensity (MFI) of the proteins secreted by those cells. For both the Neo12 NeoTCR cells and the F5 NeoTCR cells (and NeoTCR Products thereof), the polyfunctional T cell responses were strongly driven by secretion of effector proteins, including granzyme B, IFNγ, MIP1α, perforin, TNFα, TNFβ. The secretion of the following molecules from target-activated TCR-T cells were also detected: Stimulatory category included IL8; Regulatory category included sCD137, sCD40L; Chemo-attractive category included MIP-1b (FIG. 14).

These results show that, upon cognate antigen encounter, NeoTCR cells (i.e., engineered NeoTCR cells) rapidly differentiate into potent effector T cells. Engineered NeoTCR cells (i.e., NeoTCR cells) rapidly expand, secrete effector molecules such as perforin and granzyme B, and cytokines such as interferon-gamma (IFN-γ), IL-2 and TNF-alpha (TNF-α). Single-cell secretome analysis demonstrated that NeoTCR cells are highly polyfunctional (secretion of two or more cytokines or effector proteins). These results demonstrate that the autologous ex vivo engineered NeoTCR cells (i.e., the NeoTCR cells and products thereof) represent a highly personalized adoptive T cell therapy that results in the cell killing of solid and liquid tumor cells.

### Example 5

### Application of Site-specific, Highly Efficient Non-Viral Genome Engineering Technique to Multiple Cell Types

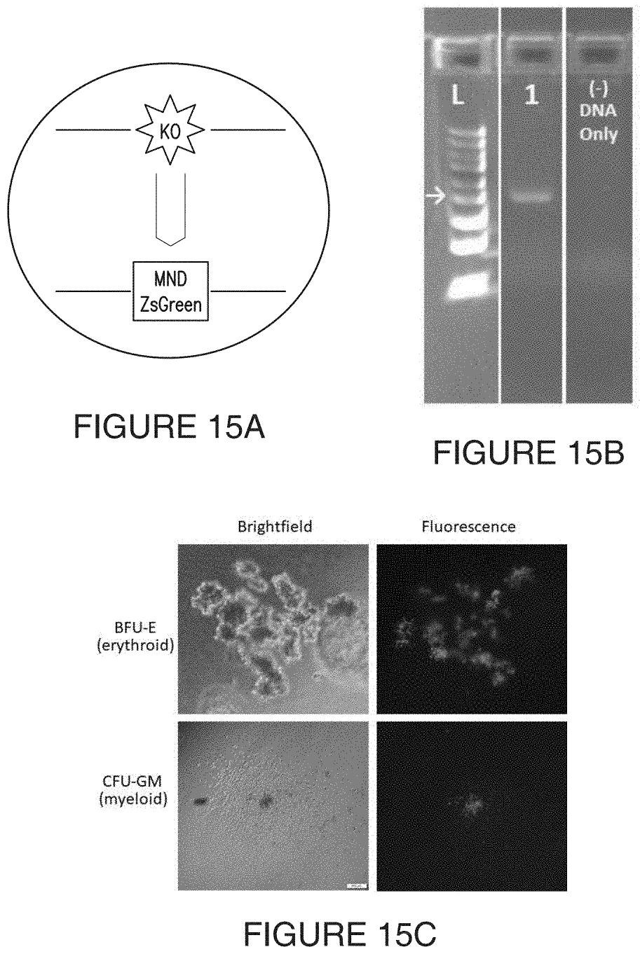

Hematopoietic Stem Cells. The non-viral precision genome engineering technique as described herein can be applied to Hematopoietic Stem Cells (HSCs) while maintaining multi-lineage potential. HSCs were engineered using a ZsGreen cassette driven by the MND promoter (FIG. 15A). In-out PCR confirmed site-specific, precise integration of the cassette (FIG. 15B). Engineered cells demonstrated proliferative capacity and multi-lineage capacity in a methylcellulose colony forming cell assay (FIG. 15C).

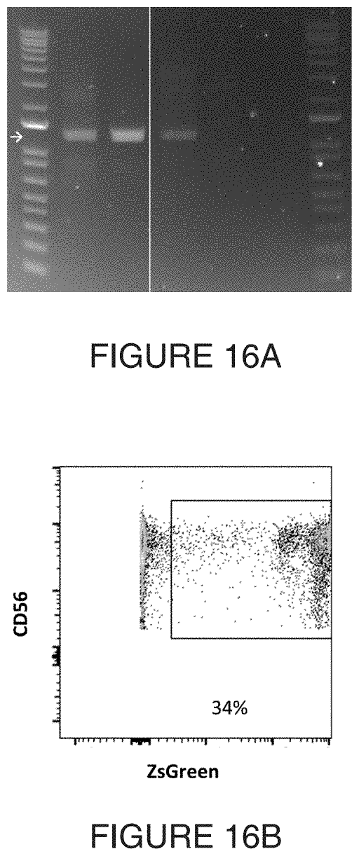

Natural Killer cells. The non-viral precision genome engineering technique as described herein can be applied to Natural Killer (NK) cells. NK cells were engineered using the same ZsGreen expression cassette shown in FIG. 15A. In-out PCR confirmed site-specific, precise integration of the cassette. Engineered T cells were used for the positive and negative controls using TCR-specific and ZsGreen-specific primers (FIG. 16A). High levels of ZsGreen expression was observed in a significant fraction of the CD3−/CD5−/CD56+ engineered cell population 11 days post-modification, (FIG. 16B).

These data evidence that the methodologies and compositions described in detail herein are capable of site-specific, highly efficient non-viral genome engineering technique that can be applied to multiple cell types including T cells, HSCs, and NKs.

Furthermore, T cell specificities can be altered to recognize neoepitopes using TCRs identified by the imPACT Isolation Technology described in PCT/US2020/17887 (which is herein incorporated by reference in its entirety).

NeoTCR cells express neoTCRs at endogenous levels, readily detectable within 3 days. Expression is maintained over time and presents no disadvantage to engineered cells.

The engineering (i.e., gene editing and manufacturing) methods described herein results in cells with a “younger” Tscm and Tcm phenotype, capable of rapidly responding to antigen by killing, proliferating, and secreting cytokines.

NeoTCR cells of stem cell memory (Tscm) and central memory (Tcm) phenotypes are the predominant T cell phenotypes resulting from the ex vivo manufacturing process described herein.

Upon contact with neoantigen-expressing surrogate tumor cells (i.e., cells expressing the cognate antigen-HLA complex), NeoTCR cells rapidly convert to effector cells to kill the tumor cells.

NeoTCR cells manufactured from both healthy donors or patients with cancer have potent antigen-specific killing and proliferative activity on contact with cognate neoantigen expressing tumor cells.

Single-cell secretome analysis demonstrated that NeoTCR cells are highly polyfunctional, even when exposed to low concentrations of cognate peptide stimulation.

Taken together, the ex vivo mechanism-of-action studies described herein demonstrate that NeoTCR cells rapidly turn into highly active tumor-killing lymphocytes upon encounter of tumor cells expressing the tumor-exclusive mutated antigen (i.e., the cognate antigen), with the potential to eradicate tumor cells throughout the body.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A method of producing a population of modified young T cells, comprising:
a) introducing via electroporation into a T cell a homologous recombination (HR) template nucleic acid sequence comprising:
i. first and second homology arms homologous to first and second target nucleic acid sequences;
ii. a T cell receptor (TCR) gene sequence positioned between the first and second homology arms;
b) recombining the HR template nucleic acid into an endogenous TCR gene locus; and
c) culturing the T cell in the presence of interleukin 2 (IL2), interleukin 7 (IL7), interleukin 15 (IL15), or any combination thereof, to thereby produce a population of modified young T cells.

2. The method of claim 1, wherein the culturing is in presence of IL7 and 11,15.

3. The method of claim 2, wherein the culturing is not in presence of IL2.

4. The method of claim 1, wherein the HR template further comprises:
a) a first P2A-coding sequence positioned upstream of the TCR gene sequence and a second P2A-coding sequence positioned downstream of the TCR gene sequence, wherein the first and second P2A-coding sequences are codon-diverged relative to each other;
b) a sequence coding for the amino acid sequence Gly Ser Gly is positioned immediately upstream of the first and second P2A-coding sequences;
c) a Furin cleavage site positioned upstream of the second P2A-coding sequence;
d) a human growth hormone (HGH) signal sequence positioned between the first 2A-coding sequence and the TCR gene sequence.

5. The method of claim 4, wherein the HR template further comprises a second TCR sequence positioned between the second P2A-coding sequence and the second homology arm and a second HGH signal sequence positioned between the second 2A-coding sequence and the second TCR gene sequence.

6. The method of claim 1, wherein the first and second homology arms are each from about 300 bases to about 2,000 bases in length.

7. The method of claim 1, wherein the HR template is a circular or linear DNA.

8. The method of claim 1, wherein the T cell is a patient-derived cell and the TCR gene sequence encodes for a TCR that recognizes a patient-derived tumor antigen.

9. The method of claim 1, wherein the TCR gene sequence is a patient-derived TCR gene sequence.

10. The method of claim 1, wherein said recombining comprises:
a) cleavage of the endogenous TCR gene locus by a nuclease; and
b) recombination of the HR template nucleic acid sequence into the endogenous TCR gene locus by homology directed repair.

11. The method of claim 1, wherein the population of modified young T cells comprises T cells that are:
a) CD45RA+, CD62L+, CD28+, CD95−, CCR7+, and CD27+;
b) CD45RA+, CD62L+, CD28+, CD95+, CD27+, CCR7+; or
c) CD45RO+, CD62L+, CD28+, CD95+, CCR7+, CD27+, CD127+.

12. The method of claim 1, wherein the population of young T cells maintains its killing activity for at least about 14 days.

13. A population of young T cells obtained by the method of claim 1.

14. A pharmaceutical composition comprising the population of young T cells obtained by the method of claim 1.

15. The pharmaceutical composition of claim 14, wherein the final formulation comprises at least about 20% of memory T stem cells (Tmsc) and central memory T cells (Tcm), collectively.

16. The pharmaceutical composition of claim 14, wherein the final formulation comprises 46% Plasma-Lyte A, 1% HSA (w/v), and 50% CryoStor CS10.

17. A method of treating cancer in a subject in need thereof, comprising administering a therapeutically effective amount of the population of modified young T cells of claim 13.

18. The method of claim 17 further comprising administering a non-myeloablative lymphodepletion regimen is administered to the subject.

19. The method of claim 17, wherein the cancer is a tumor selected from the group consisting of follicular lymphoma, leukemia, multiple myeloma, melanoma, thoracic cancer, lung cancer, ovarian cancer, breast cancer, pancreatic cancer, head and neck cancer, prostate cancer, gynecological cancer, central nervous system cancer, cutaneous cancer, HPV+ cancer, esophageal cancer, thyroid cancer, gastric cancer, hepatocellular cancer, cholangiocarcinomas, renal cell cancers, testicular cancer, sarcomas, and colorectal cancer.

20. The method of claim 17, wherein the population of young T cells maintains its killing activity for at least about 60 days, and engrafts into the subject following the administration.
